SEQUENCE LISTING (20 sequences)

SEQ ID NO: 1 - DNA, 20 bp, Homo sapiens
tatgggaaggagtcgttgag

SEQ ID NO: 2 - DNA, 20 bp, Homo sapiens
ctgaatcactccttgccaac

SEQ ID NO: 3 - DNA, 20 bp, Homo sapiens
caaaatgatctgactactcc

SEQ ID NO: 4 - DNA, 20 bp, Homo sapiens
tgaccagtgctgtatgtatc

SEQ ID NO: 5 - DNA, 20 bp, Homo sapiens
tctcacctgataccagattc

SEQ ID NO: 6 - DNA, 20 bp, Homo sapiens
tctctccttaatgcctctat

SEQ ID NO: 7 - DNA, 20 bp, Homo sapiens
actgctgcgggttcctaaag

SEQ ID NO: 8 - DNA, 21 bp, Homo sapiens
ggaagattcgattcaacaagg

SEQ ID NO: 9 - DNA, 19 bp, Homo sapiens
ggtaactatgaatctcatc

SEQ ID NO: 10 - DNA, 20 bp, Homo sapiens
aaaaagcagagaaagcaggg

SEQ ID NO: 11 - DNA, 20 bp, Homo sapiens
agatgatctctgagatgccc

SEQ ID NO: 12 - DNA, 20 bp, Homo sapiens
ccagggtttgtctaccaaag

SEQ ID NO: 13 - DNA, 19 bp, Homo sapiens
aatcacttaaaacaagcag

SEQ ID NO: 14 - DNA, 20 bp, Homo sapiens
cacatacctctacctctagc

SEQ ID NO: 15 - DNA, 19 bp, Homo sapiens
ttccctagtggagcagtgg

SEQ ID NO: 16 - DNA, 20 bp, Homo sapiens
ctttcttcgcaaatgggtgg

SEQ ID NO: 17 - DNA, 20 bp, Homo sapiens
gcactcatcgccacttaatg

SEQ ID NO: 18 - DNA, 20 bp, Homo sapiens
gaacagctaaaccagaacag

SEQ ID NO: 19 - DNA, 20 bp, Homo sapiens
atcactcttatttctccccc

SEQ ID NO: 20 - DNA, 20 bp, Homo sapiens
tgagtcactgtgctaaggag

(19) United States
(12) Patent Application Publication
(10) Pub. No.: US 2020/0102617 A1
(43) Pub. Date: Apr. 2, 2020

(54) Improved Methods For Assessing Risk of Developing Breast Cancer

(71) Applicant: Genetic Technologies Limited, Fitzroy, Victoria (AU)

(72) Inventor: Richard Allman, Wyndham Vale, Victoria (AU)

(21) Appl. No.: 16/480,516

(22) PCT Filed: Jan. 23, 2018
(86) PCT No.: PCT/AU2018/050041

§ 371 Date: Jul. 24, 2019

(30) Foreign Application Priority Data
Jan. 24, 2017 (AU) 2017900208

(51) Int. Cl. C12Q 1/6886; G16B 20/20; G16B 40/20; G16H 50/30; G16H 50/70

(52) U.S. Cl. CPC C12Q 1/6886; G16B 20/20; G16B 40/20; A61K 45/06; G16H 50/70; C12Q 2600/156; G16H 50/30

(57) ABSTRACT

The present disclosure relates to methods and systems for assessing the risk of a human female subject for developing a breast cancer. In particular, the present disclosure relates to combining simplified clinical risk assessment and genetic risk assessment to improve risk analysis.

### CROSS-REFERENCE TO RELATED APPLICATIONS

This application is a § 371 national stage of PCT International Application No. PCT/AU2018/050041, filed Jan. 23, 2018, claiming priority of Australian Patent Application No. AU2017900208, filed Jan. 24, 2017, the contents of each of which are hereby incorporated by reference into the application.

### REFERENCE TO A SEQUENCE LISTING

This application incorporates-by-reference nucleotide and/or amino acid sequences which are present in the file named “190724_5938_91058_Sequence_Listing_SC.txt”, which is 4 kilobytes in size, and which was created Jul. 24, 2019 in the IBM-PC machine format, having an operating system compatibility with MS-Windows, which is contained in the text file filed Jul. 24, 2019 as part of this application.

### TECHNICAL FIELD

The present disclosure relates to methods and systems for assessing the risk of a human female subject for developing a breast cancer. In particular, the present disclosure relates to combining simplified clinical risk assessment and genetic risk assessment to improve risk analysis.

### BACKGROUND OF THE INVENTION

It is estimated that in the USA approximately one in eight women will develop breast cancer in their lifetime. In 2013 it was predicted that over 230,000 women would be diagnosed with invasive breast cancer and almost 40,000 would die from the disease (ACS Breast Cancer Facts & Figures 2013-14). There is therefore a compelling reason to predict which women will develop disease, and to apply measures to prevent it.

A wide body of research has focused on phenotypic risk factors including age, family history, reproductive history, and benign breast disease. Various combinations of these risk factors have been compiled into the two most commonly used risk prediction algorithms; the Gail Model (appropriate for the general population) (also known as the Breast Cancer Risk Assessment Tool: BCRAT) and the Tyrer-Cuzick Model (appropriate for women with a stronger family history).

These risk prediction algorithms rely largely on self-reported clinical information which is usually obtained by questionnaire. In some instances, relevant clinical information is not provided. This is to be expected, as some questions are reliant on memory from decades' past (first menses), while others require a level of medical sophistication on the part of the patient and/or actual pathology reports (atypical hyperplasia). Furthermore, for those entering an answer rather than ‘unknown’, it brings in to the question the accuracy of data set being entered into the algorithm. For example, whether or not atypical hyperplasia was present is an important factor in breast cancer risk assessment (Relative Risk >4.0).

Recent, commercially available tests for assessing the risk of developing breast cancer discuss predicting breast cancer risk by combining clinical and genetic risk scores. However, the clinical risk assessment components of these tests are subject to the above referenced limitations of self-reported clinical information. Accordingly, there is the need for improved breast cancer risk assessment tests.

### SUMMARY OF THE INVENTION

The present inventors have identified a simplified clinical risk assessment that can be combined with a genetic risk assessment to provide an improved method of assessing breast cancer risk in female subjects.

In an embodiment, the present disclosure relates to a method for assessing the risk of a human female subject for developing breast cancer comprising:

performing a clinical risk assessment of the female subject, wherein the clinical risk assessment is based only on two or all of the female subjects age, family history of breast cancer and ethnicity;

performing a genetic risk assessment of the female subject, wherein the genetic risk assessment involves detecting, in a biological sample derived from the female subject, the presence at least two single nucleotide polymorphisms known to be associated with breast cancer; and

combining the clinical risk assessment with the genetic risk assessment to obtain the risk of a human female subject for developing breast cancer.

For example, the genetic risk assessment can comprise detecting the presence of at least three, five, 10, 20, 30, 40, 50, 60, 70, 80 single nucleotide polymorphisms known to be associated with breast cancer.

In an embodiment, single nucleotide polymorphisms are individually tested for association with breast cancer by logistic regression under a log additive model with no covariates.

In another embodiment, the single nucleotide polymorphisms are selected from a group consisting of rs2981582, rs3803662, rs889312, rs13387042, rs13281615, rs4415084, rs3817198, rs4973768, rs6504950 and rs11249433, or a single nucleotide polymorphism in linkage disequilibrium with one or more thereof. In another embodiment, the single nucleotide polymorphisms are selected from Table 6 or a single nucleotide polymorphism in linkage disequilibrium with one or more thereof.

In another embodiment, the genetic risk assessment may comprise detecting at least 72 single nucleotide polymorphisms associated with breast cancer, wherein at least 67 of the single nucleotide polymorphisms are selected from Table 7, or a single nucleotide polymorphism in linkage disequilibrium with one or more thereof, and the remaining single nucleotide polymorphisms are selected from Table 6, or a single nucleotide polymorphism in linkage disequilibrium with one or more thereof.

In an embodiment, the genetic risk assessment may be varied based on the ethnicity of the female subject being assessed. For example, when the female subject is Caucasian, the genetic risk assessment comprises detecting at least 72 single nucleotide polymorphisms shown in Table 9, or a single nucleotide polymorphism in linkage disequilibrium with one or more thereof. In another example, when the female subject is Caucasian, the genetic risk assessment comprises detecting at least the 77 single nucleotide polymorphisms shown in Table 9, or a single nucleotide polymorphism in linkage disequilibrium with one or more thereof. In another example, when the female subject is Negroid or African-American, the genetic risk assessment comprises detecting at least 74 single nucleotide polymorphisms shown in Table 10, or a single nucleotide polymorphism in linkage disequilibrium with one or more thereof. In another example, when the female subject is Negroid or African-American, the genetic risk assessment comprises detecting at least the 78 single nucleotide polymorphisms shown in Table 10, or a single nucleotide polymorphism in linkage disequilibrium with one or more thereof. In another example, when the female subject is Hispanic, the genetic risk assessment comprises detecting at least 78 single nucleotide polymorphisms shown in Table 11, or a single nucleotide polymorphism in linkage disequilibrium with one or more thereof. In another example, when the female subject is Hispanic, the genetic risk assessment comprises detecting at least the 82 single nucleotide polymorphisms shown in Table 11, or a single nucleotide polymorphism in linkage disequilibrium with one or more thereof.

In an embodiment, a single nucleotide polymorphism in linkage disequilibrium has linkage disequilibrium above 0.9. In another embodiment, a single nucleotide polymorphism in linkage disequilibrium has linkage disequilibrium above 1.

In an embodiment, the clinical risk assessment is based only on the female subjects age and family history of breast cancer. In another embodiment, the clinical risk assessment is based only on the female subjects age, family history of breast cancer and ethnicity.

In another embodiment, combining the clinical risk assessment with the genetic risk assessment comprises multiplying the risk assessments to provide the risk score.

In an embodiment, the results of the clinical risk assessment indicate that the female subject should be subjected to more frequent screening and/or prophylactic anti-breast cancer therapy.

In another embodiment, if it is determined the subject has a risk of developing breast cancer, the subject is more likely to be responsive to estrogen inhibition therapy than non-responsive.

In an embodiment, the breast cancer may be estrogen receptive positive or estrogen receptor negative.

In another embodiment, the methods of the present disclosure may be incorporated into a method for determining the need for routine diagnostic testing of a human female subject for breast cancer.

In an embodiment, performing the clinical risk assessment uses a model which calculates the absolute risk of developing breast cancer. For example, the absolute risk of developing breast cancer may be calculated using breast cancer incidence rates and takes into account the competing risk of dying from other causes apart from breast cancer.

In another embodiment, the clinical risk assessment provides a 5-year absolute risk of developing breast cancer. In another embodiment, the clinical risk assessment provides a 10-year absolute risk of developing breast cancer.

In another embodiment, performing the clinical risk assessment uses a model which calculates the lifetime risk of developing breast cancer. In an embodiment, a risk score greater than about 20% lifetime risk indicates that the subject should be enrolled in a screening breast MRI and mammography program.

In another embodiment, the present disclosure encompasses a method of screening for breast cancer in a human female subject, the method comprising assessing the risk of the subject for developing breast cancer using the methods of the present disclosure and routinely screening for breast cancer in the subject if they are assessed as having a risk for developing breast cancer.

In another embodiment, the methods of the present disclosure may be incorporated into a method for determining the need of a human female subject for prophylactic anti-breast cancer therapy. In an embodiment, a risk score greater than about 1.66% 5-year risk indicates that estrogen receptor therapy should be offered to the subject.

In another embodiment, the present disclosure encompasses a method for preventing or reducing the risk of breast cancer in a human female subject, the method comprising assessing the risk of the subject for developing breast cancer using the methods according to the present disclosure, and administering an anti-breast cancer therapy to the subject if they are assessed as having a risk for developing breast cancer. In an embodiment, the therapy inhibits estrogen.

In another embodiment, the present disclosure encompasses an anti-breast cancer therapy for use in preventing breast cancer in a human female subject at risk thereof, wherein the subject is assessed as having a risk for developing breast cancer according to the methods of the present disclosure.

In another embodiment, the present disclosure encompasses a method for stratifying a group of human female subjects for a clinical trial of a candidate therapy, the method comprising assessing the individual risk of the subjects for developing breast cancer using the methods according to the present disclosure, and using the results of the assessment to select subjects more likely to be responsive to the therapy.

In another embodiment, the present disclosure encompasses a computer implemented method for assessing the risk of a human female subject for developing breast cancer, the method operable in a computing system comprising a processor and a memory, the method comprising:

receiving clinical risk data and genetic risk data for the female subject, wherein the clinical and genetic risk data was obtained by a method according to the present disclosure;

processing the data to combine the clinical risk data with the genetic risk data to obtain the risk of a human female subject for developing breast cancer;

outputting the risk of a human female subject for developing breast cancer.

In another embodiment, the present disclosure encompasses a system for assessing the risk of a human female subject for developing breast cancer comprising: system instructions for performing a clinical risk assessment and a genetic risk assessment of the female subject according to the present disclosure; and

system instructions for combining the clinical risk assessment with the genetic risk assessment to obtain the risk of a human female subject for developing breast cancer.

Any example herein shall be taken to apply mutatis mutandis to any other example unless specifically stated otherwise.

The present disclosure is not to be limited in scope by the specific examples described herein, which are intended for the purpose of exemplification only. Functionally-equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The disclosure is hereinafter described by way of the following non-limiting Examples and with reference to the accompanying drawings.

### BRIEF DESCRIPTION OF THE ACCOMPANYING DRAWINGS

FIG. 1: Depicts patients integrated 5-year risk using the Gail Model for the clinical risk assessment.

FIG. 2: (a) Box and whisker plot of 2,282 US patient 5-year risk scores using the simple clinical risk (SCR) model plus SNPs, or the Gail Model plus SNPs. The circles represent outliers. (b) log-transformed values of the 5-year distributions and the t-test results. The t-test indicates that there is no difference in mean values between SCR plus SNP and Gail plus SNP scores (P>0.05).

FIG. 3: ROC plots of risk prediction for SNPs only, SCR model only, or SCR plus risk SNP in (a) African American, (b) Caucasian, and (c) Hispanic women. A reference line of random risk prediction is also shown.

FIG. 4: Depicts the patients absolute 5-year risk using the SCR model.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., oncology, breast cancer analysis, molecular genetics, risk assessment and clinical studies).

Unless otherwise indicated, the molecular, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes I and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

As used herein, the term “about”, unless stated to the contrary, refers to +/−10%, more preferably +/−5%, more preferably +/−1%, of the designated value.

The methods of the present disclosure can be used to assess risk of a human female subject developing breast cancer. As used herein, the term “breast cancer” encompasses any type of breast cancer that can develop in a female subject. For example, the breast cancer may be characterised as Luminal A (ER+ and/or PR+, HER2−, low Ki67), Luminal B (ER+ and/or PR+, HER2+(or HER2− with high Ki67), Triple negative/basal-like (ER−, PR−, HER2−) or HER2 type (ER−, PR−, HER2+). In another example, the breast cancer may be resistant to therapy or therapies such as alkylating agents, platinum agents, taxanes, vinca agents, anti-estrogen drugs, aromatase inhibitors, ovarian suppression agents, endocrine/hormonal agents, bisphophonate therapy agents or targeted biological therapy agents. As used herein, “breast cancer” also encompasses a phenotype that displays a predisposition towards developing breast cancer in an individual. A phenotype that displays a predisposition for breast cancer, can, for example, show a higher likelihood that the cancer will develop in an individual with the phenotype than in members of a relevant general population under a given set of environmental conditions (diet, physical activity regime, geographic location, etc.).

As used herein, “biological sample” refers to any sample comprising nucleic acids, especially DNA, from or derived from a human patient, e.g., bodily fluids (blood, saliva, urine etc.), biopsy, tissue, and/or waste from the patient. Thus, tissue biopsies, stool, sputum, saliva, blood, lymph, or the like can easily be screened for SNPs, as can essentially any tissue of interest that contains the appropriate nucleic acids. In one embodiment, the biological sample is a cheek cell sample. These samples are typically taken, following informed consent, from a patient by standard medical laboratory methods. The sample may be in a form taken directly from the patient, or may be at least partially processed (purified) to remove at least some non-nucleic acid material.

A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele “negatively” correlates with a trait when it is linked to it and when presence of the allele is an indicator that a trait or trait form will not occur in an individual comprising the allele.

A marker polymorphism or allele is “correlated” or “associated” with a specified phenotype (breast cancer susceptibility, etc.) when it can be statistically linked (positively or negatively) to the phenotype. Methods for determining whether a polymorphism or allele is statistically linked are known to those in the art. That is, the specified polymorphism occurs more commonly in a case population (e.g., breast cancer patients) than in a control population (e.g., individuals that do not have breast cancer). This correlation is often inferred as being causal in nature, but it need not be, simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies (Slatkin and Excoffier, 1996). LD values according to the present disclosure for neighbouring genotypes/loci are selected above 0.1, preferably, above 0.2, more preferable above 0.5, more preferably, above 0.6, still more preferably, above 0.7, preferably, above 0.8, more preferably above 0.9, ideally about 1.0.

Another way one of skill in the art can readily identify SNPs in linkage disequilibrium with the SNPs of the present disclosure is determining the LOD score for two loci. LOD stands for “logarithm of the odds”, a statistical estimate of whether two genes, or a gene and a disease gene, are likely to be located near each other on a chromosome and are therefore likely to be inherited. A LOD score of between about 2-3 or higher is generally understood to mean that two genes are located close to each other on the chromosome. Various examples of SNPs in linkage disequilibrium with the SNPs of the present disclosure are shown in Tables 1 to 4. The present inventors have found that many of the SNPs in linkage disequilibrium with the SNPs of the present disclosure have a LOD score of between about 2-50. Accordingly, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, SNPs in linkage disequilibrium with the SNPs of the present disclosure can have a specified genetic recombination distance of less than or equal to about 20 centimorgan (cM) or less. For example, 15 cM or less, 10 cM or less, 9 cM or less, 8 cM or less, 7 cM or less, 6 cM or less, 5 cM or less, 4 cM or less, 3 cM or less, 2 cM or less, 1 cM or less, 0.75 cM or less, 0.5 cM or less, 0.25 cM or less, or 0.1 cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, SNPs in linkage disequilibrium with the SNPs of the present disclosure are within at least 100 kb (which correlates in humans to about 0.1 cM, depending on local recombination rate), at least 50 kb, at least 20 kb or less of each other.

For example, one approach for the identification of surrogate markers for a particular SNP involves a simple strategy that presumes that SNPs surrounding the target SNP are in linkage disequilibrium and can therefore provide information about disease susceptibility. Thus, as described herein, surrogate markers can therefore be identified from publicly available databases, such as HAPMAP, by searching for SNPs fulfilling certain criteria which have been found in the scientific community to be suitable for the selection of surrogate marker candidates (see, for example, the legends of Tables 1 to 4).

“Allele frequency” refers to the frequency (proportion or percentage) at which an allele is present at a locus within an individual, within a line or within a population of lines. For example, for an allele “A,” diploid individuals of genotype “AA,” “Aa,” or “aa” have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the minor allele frequency (MAF). MAF refers to the frequency at which the least common allele occurs in a given population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose, e.g., identifying an individual with a specified genotype (e.g., risk of developing breast cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

As used herein, “Receiver operating characteristic curves” (ROC) refer to a graphical plot of the sensitivity vs. (1−specificity) for a binary classifier system as its discrimination threshold is varied. The ROC can also be represented equivalently by plotting the fraction of true positives (TPR=true positive rate) vs. the fraction of false positives (FPR=false positive rate). Also known as a Relative Operating Characteristic curve, because it is a comparison of two operating characteristics (TPR & FPR) as the criterion changes. ROC analysis provides tools to select possibly optimal models and to discard suboptimal ones independently from (and prior to specifying) the cost context or the class distribution. Methods of using in the context of the disclosure will be clear to those skilled in the art.

As used herein, the term “combining the clinical risk assessment with the genetic risk assessment to obtain the risk” refers to any suitable mathematical analysis relying on the results of the two assessments. For example, the results of the clinical risk assessment and the genetic risk assessment may be added, more preferably multiplied.

As used herein, the terms “routinely screening for breast cancer” and “more frequent screening” are relative terms, and are based on a comparison to the level of screening recommended to a subject who has no identified risk of developing breast cancer.

### Clinical Risk Assessment

In an embodiment, the clinical risk assessment procedure includes obtaining clinical information from a female subject. In other embodiments these details have already been determined (such as in the subjects medical records).

In an embodiment, the clinical risk assessment at least takes into consideration the age of the female. In another embodiment, the clinical risk assessment is based only on the female subjects age and family history of breast cancer. In this embodiment, the clinical risk assessment can optionally also take ethnicity into consideration. Accordingly, in another embodiment, the clinical risk assessment is based only on the female subjects family history of breast cancer and ethnicity. In another embodiment, the clinical risk assessment is based only on the female subjects age and ethnicity. In another embodiment, the clinical risk assessment is based only on the female subjects age, family history of breast cancer and ethnicity.

“Family history of breast cancer” is used in the context of the present disclosure to refer to the history of breast cancer amongst the female subjects first and/or second degree relatives. For example, “family history of breast cancer” can be used to refer to the history of breast cancer amongst only first degree relatives. Put another way, the clinical risk assessment procedure can take into consideration the female subjects family history of breast cancer amongst first degree relatives. In the context of the present disclosure, a “first degree relative” is a family member who shares about 50 percent of their genes with the female subject. Examples of first degree relatives include parents, offspring, and full-siblings. A “second-degree relative” is a family member who shares about 25 percent of their genes with the female subject. Examples of second degree relatives include uncles, aunts, nephews, nieces, grandparents, grandchildren, and half-siblings.

Accordingly, in an embodiment, the clinical risk assessment is based only on the age of the female subject and known history of breast cancer among first degree relatives. In another embodiment, the clinical risk assessment is based on the age of the female subject, known history of breast cancer among first degree relatives and ethnicity.

As used herein, “based on” means that values are assigned to, for example, the subjects age and family history of breast cancer, but then any suitable calculations are conducted to determine clinical risk.

Clinical information can be self-reported by the female subject. For example, the subject may complete a questionnaire designed to obtain clinical information such as age, history of breast cancer among first degree relatives and ethnicity. In another example, subject to obtaining informed consent from the female subject, clinical information can be obtained from medical records by interrogating a relevant database comprising the clinical information.

In an embodiment, the clinical risk assessment procedure provides an estimate of the risk of the human female subject developing breast cancer during the next 5-year period (i.e. 5-year risk).

In another embodiment, the clinical risk assessment procedure provides an estimate of the risk of the human female subject developing breast cancer up to age 90 (i.e. lifetime risk In another embodiment, performing the clinical risk assessment uses a model which calculates the absolute risk of developing breast cancer. For example, the absolute risk of developing breast cancer can be calculated using cancer incidence rates while accounting for the competing risk of dying from other causes apart from breast cancer.

In an embodiment, the clinical risk assessment provides a 5-year absolute risk of developing breast cancer. In another embodiment, the clinical risk assessment provides a 10-year absolute risk of developing breast cancer.

### Genetic Risk Assessment

In an embodiment the genetic risk assessment is performed by analysing the genotype of the subject at 2 or more loci for single nucleotide polymorphisms associated with breast cancer. Various exemplary single nucleotide polymorphisms associated with breast cancer are discussed in the present disclosure. These single nucleotide polymorphisms vary in terms of penetrance and many would be understood by those of skill in the art to be low penetrance single nucleotide polymorphisms.

The term “penetrance” is used in the context of the present disclosure to refer to the frequency at which a particular single nucleotide polymorphisms genotype manifests itself within female subjects with breast cancer. “High penetrance” single nucleotide polymorphisms will almost always be apparent in a female subject with breast cancer while “low penetrance” single nucleotide polymorphisms will only sometimes be apparent. In an embodiment SNPs assessed as part of a genetic risk assessment according to the present disclosure are low penetrance SNPs.

As the skilled addressee will appreciate, each SNP which increases the risk of developing breast cancer has an odds ratio of association with breast cancer of greater than 1.0. In an embodiment, the odds ratio is greater than 1.02. Each SNP which decreases the risk of developing breast cancer has an odds ratio of association with breast cancer of less than 1.0. In an embodiment, the odds ratio is less than 0.98. Examples of such SNPs include, but are not limited to, those provided in Tables 6 to 11, or a single nucleotide polymorphism in linkage disequilibrium with one or more thereof. In an embodiment the genetic risk assessment involves assessing SNPs associated with increased risk of developing breast cancer. In another embodiment, the genetic risk assessment involves assessing SNPs associated with decreased risk of developing breast cancer. In another embodiment, the genetic risk assessment involves assessing SNPs associated with an increased risk of developing breast cancer and SNPs associated with a decreased risk of developing breast cancer.

In an embodiment, the genetic risk assessment is performed by analysing the genotype of the subject at two, three, four, five, six, seven, eight, nine, 10 or more loci for single nucleotide polymorphisms associated with breast cancer. Exemplary, single nucleotide polymorphisms relevant for the assessment of breast cancer risk include rs2981582, rs3803662, rs889312, rs13387042, rs13281615, rs4415084, rs3817198, rs4973768, rs6504950 and rs11249433, or a single nucleotide polymorphism in linkage disequilibrium with one or more thereof.

In another embodiment, the genetic risk assessment is performed by analysing the genotype of the subject at 20, 30, 40, 50, 60, 70, 80 or more loci for single nucleotide polymorphisms associated with breast cancer.

In an embodiment, the genetic risk assessment is performed by analysing the genotype of the subject at 72 or more loci for single nucleotide polymorphisms associated with breast cancer.

In an embodiment, when performing the methods of the present disclosure to assess risk of breast cancer, at least 67 of the single nucleotide polymorphisms are selected from Table 7 or a single nucleotide polymorphism in linkage disequilibrium with one or more thereof and the remaining single nucleotide polymorphisms are selected from Table 6, or a single nucleotide polymorphism in linkage disequilibrium with one or more thereof. In another embodiment, when performing the methods of the present disclosure at least 68, at least 69, at least 70 of the single nucleotide polymorphisms are selected from Table 7 or a single nucleotide polymorphism in linkage disequilibrium with one or more thereof and the remaining single nucleotide polymorphisms are selected from Table 6, or a single nucleotide polymorphism in linkage disequilibrium with one or more thereof. In one embodiment, at least 72, at least 73, at least 74, at least 75, at least 76, at least 77, at least 78, at least 79, at least 80, at least 81, at least 82, at least 83, at least 84, at least 85, at least 86, at least 87, at least 88 of the single nucleotide polymorphisms shown in Table 6, or a single nucleotide polymorphism in linkage disequilibrium with one or more thereof are assessed. In further embodiments, at least 67, at least 68, at least 69, at least 70 of the single nucleotide polymorphisms shown in Table 7, or a single nucleotide polymorphism in linkage disequilibrium with one or more thereof are assessed. In further embodiments, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88 single nucleotide polymorphisms are assessed, wherein at least 67, at least 68, at least 69, at least 70 single nucleotide polymorphisms shown in Table 7, or a single nucleotide polymorphism in linkage disequilibrium with one or more thereof are assessed, with any remaining single nucleotide polymorphisms being selected from Table 6, or a single nucleotide polymorphism in linkage disequilibrium with one or more thereof.

SNPs in linkage disequilibrium with those specifically mentioned herein are easily identified by those of skill in the art. Examples of such SNPs include rs1219648 and rs2420946 which are in strong linkage disequilibrium with rs2981582 (further possible examples provided in Table 1), rs12443621 and rs8051542 which are in strong linkage disequilibrium with SNP rs3803662 (further possible examples provided in Table 2), and rs10941679 which is in strong linkage disequilibrium with SNP rs4415084 (further possible examples provided in Table 3). In addition, examples of SNPs in linkage disequilibrium with rs13387042 are provided in Table 4. Such linked polymorphisms for the other SNPs listed in Table 6 can very easily be identified by the skilled person using the HAPMAP database.

TABLE 1
Surrogate markers for SNP rs2981582. Markers with a r2
greater than 0.05 to rs2981582 in the HAPMAP dataset
(http://hapmap.ncbi.nlm.nih.gov) in a 1 Mbp interval
flanking the marker was selected. Shown is the name of the
correlated SNP, values for r2 and D′ to rs2981582 and the
corresponding LOD value, as well as the position of the
surrogate marker in NCB Build 36.

| DbSNP | | Correlated | | | | |
|---|---|---|---|---|---|---|
| rsID | Position | SNP | Location | D′ | r² | LOD |
| rs2981582 | 123342307 | rs3135715 | 123344716 | 1.000 | 0.368 | 15.02 |
| rs2981582 | 123342307 | rs7899765 | 123345678 | 1.000 | 0.053 | 2.44 |
| rs2981582 | 123342307 | rs1047111 | 123347551 | 0.938 | 0.226 | 9.11 |
| rs2981582 | 123342307 | rs1219639 | 123348302 | 1.000 | 0.143 | 6.53 |
| rs2981582 | 123342307 | rs10886955 | 123360344 | 0.908 | 0.131 | 5.42 |
| rs2981582 | 123342307 | rs1631281 | 123380775 | 0.906 | 0.124 | 5.33 |
| rs2981582 | 123342307 | rs3104685 | 123381354 | 0.896 | 0.108 | 4.58 |
| rs2981582 | 123342307 | rs1909670 | 123386718 | 1.000 | 0.135 | 6.12 |
| rs2981582 | 123342307 | rs7917459 | 123392364 | 1.000 | 0.135 | 6.42 |
| rs2981582 | 123342307 | rs17102382 | 123393846 | 1.000 | 0.135 | 6.42 |
| rs2981582 | 123342307 | rs10788196 | 123407625 | 1.000 | 0.202 | 9.18 |
| rs2981582 | 123342307 | rs2935717 | 123426236 | 0.926 | 0.165 | 7.30 |
| rs2981582 | 123342307 | rs3104688 | 123426455 | 0.820 | 0.051 | 2.07 |
| rs2981582 | 123342307 | rs4752578 | 123426514 | 1.000 | 0.106 | 5.15 |
| rs2981582 | 123342307 | rs1696803 | 123426940 | 0.926 | 0.168 | 7.33 |
| rs2981582 | 123342307 | rs12262574 | 123428112 | 1.000 | 0.143 | 7.39 |
| rs2981582 | 123342307 | rs4752579 | 123431182 | 1.000 | 0.106 | 5.15 |
| rs2981582 | 123342307 | rs12358208 | 123460953 | 0.761 | 0.077 | 2.46 |
| rs2981582 | 123342307 | rs17102484 | 123462020 | 0.758 | 0.065 | 2.39 |
| rs2981582 | 123342307 | rs2936859 | 123469277 | 0.260 | 0.052 | 1.56 |
| rs2981582 | 123342307 | rs10160140 | 123541979 | 0.590 | 0.016 | 0.40 |

TABLE 2
Surrogate markers for SNP rs3803662. Markers with a r2 greater than 0.05
to rs3803662 in the HAPMAP dataset (http://hapmap.ncbi.nlm.nih.gov) in
a 1 Mbp interval flanking the marker was selected. Shown is the name of
the correlated SNP, values for r2 and D′ to rs3803662 and the
corresponding LOD value, as well as the position of the surrogate marker
in NCB Build 36.

| DbSNP | | Correlated | | | | |
|---|---|---|---|---|---|---|
| rsID | Position | SNP | Location | D′ | r² | LOD |
| rs3803662 | 51143842 | rs4784227 | 51156689 | 0.968 | 0.881 | 31.08 |
| rs3803662 | 51143842 | rs3112572 | 51157948 | 1.000 | 0.055 | 1.64 |
| rs3803662 | 51143842 | rs3104747 | 51159425 | 1.000 | 0.055 | 1.64 |
| rs3803662 | 51143842 | rs3104748 | 51159860 | 1.000 | 0.055 | 1.64 |
| rs3803662 | 51143842 | rs3104750 | 51159990 | 1.000 | 0.055 | 1.64 |
| rs3803662 | 51143842 | rs3104758 | 51166534 | 1.000 | 0.055 | 1.64 |
| rs3803662 | 51143842 | rs3104759 | 51167030 | 1.000 | 0.055 | 1.64 |
| rs3803662 | 51143842 | rs9708611 | 51170166 | 1.000 | 0.169 | 4.56 |
| rs3803662 | 51143842 | rs12935019 | 51170538 | 1.000 | 0.088 | 4.04 |
| rs3803662 | 51143842 | rs4784230 | 51175614 | 1.000 | 0.085 | 4.19 |
| rs3803662 | 51143842 | rs11645620 | 51176454 | 1.000 | 0.085 | 4.19 |
| rs3803662 | 51143842 | rs3112633 | 51178078 | 1.000 | 0.085 | 4.19 |
| rs3803662 | 51143842 | rs3104766 | 51182036 | 0.766 | 0.239 | 7.55 |
| rs3803662 | 51143842 | rs3104767 | 51182239 | 0.626 | 0.167 | 4.88 |
| rs3803662 | 51143842 | rs3112625 | 51183053 | 0.671 | 0.188 | 5.62 |
| rs3803662 | 51143842 | rs12920540 | 51183114 | 0.676 | 0.195 | 5.84 |
| rs3803662 | 51143842 | rs3104774 | 51187203 | 0.671 | 0.188 | 5.62 |
| rs3803662 | 51143842 | rs7203671 | 51187646 | 0.671 | 0.188 | 5.62 |
| rs3803662 | 51143842 | rs3112617 | 51189218 | 0.666 | 0.177 | 5.44 |
| rs3803662 | 51143842 | rs11075551 | 51189465 | 0.666 | 0.177 | 5.44 |
| rs3803662 | 51143842 | rs12929797 | 51190445 | 0.676 | 0.19 | 5.87 |
| rs3803662 | 51143842 | rs3104780 | 51191415 | 0.671 | 0.184 | 5.65 |
| rs3803662 | 51143842 | rs12922061 | 51192501 | 0.832 | 0.631 | 19.14 |
| rs3803662 | 51143842 | rs3112612 | 51192665 | 0.671 | 0.184 | 5.65 |
| rs3803662 | 51143842 | rs3104784 | 51193866 | 0.666 | 0.177 | 5.44 |
| rs3803662 | 51143842 | rs12597685 | 51195281 | 0.671 | 0.184 | 5.65 |
| rs3803662 | 51143842 | rs3104788 | 51196004 | 0.666 | 0.177 | 5.44 |
| rs3803662 | 51143842 | rs3104800 | 51203877 | 0.625 | 0.17 | 4.99 |
| rs3803662 | 51143842 | rs3112609 | 51206232 | 0.599 | 0.163 | 4.86 |
| rs3803662 | 51143842 | rs3112600 | 51214089 | 0.311 | 0.016 | 0.57 |
| rs3803662 | 51143842 | rs3104807 | 51215026 | 0.302 | 0.014 | 0.52 |
| rs3803662 | 51143842 | rs3112594 | 51229030 | 0.522 | 0.065 | 1.56 |
| rs3803662 | 51143842 | rs4288991 | 51230665 | 0.238 | 0.052 | 1.53 |
| rs3803662 | 51143842 | rs3104820 | 51233304 | 0.528 | 0.069 | 1.60 |
| rs3803662 | 51143842 | rs3104824 | 51236594 | 0.362 | 0.067 | 1.93 |
| rs3803662 | 51143842 | rs3104826 | 51237406 | 0.362 | 0.067 | 1.93 |
| rs3803662 | 51143842 | rs3112588 | 51238502 | 0.354 | 0.062 | 1.80 |

TABLE 3
Surrogate markers for SNP rs4415084. Markers with a r2 greater than 0.05
to rs4415084 in the HAPMAP dataset (http://hapmap.ncbi.nlm.nih.gov) in a
1 Mbp interval flanking the marker was selected. Shown is the name of the
correlated SNP, values for r2 and D′ to rs4415084 and the corresponding
LOD value, as well as the position of the surrogate marker in NCB Build 36.

| DbSNP | | Correlated | | | | |
|---|---|---|---|---|---|---|
| rsID | Position | SNP | Location | D′ | r² | LOD |
| rs4415084 | 44698272 | rs12522626 | 44721455 | 1.000 | 1.0 | 47.37 |
| rs4415084 | 44698272 | rs4571480 | 44722945 | 1.000 | 0.976 | 40.54 |
| rs4415084 | 44698272 | rs6451770 | 44727152 | 1.000 | 0.978 | 44.88 |
| rs4415084 | 44698272 | rs920328 | 44734808 | 1.000 | 0.893 | 39.00 |
| rs4415084 | 44698272 | rs920329 | 44738264 | 1.000 | 1.0 | 47.37 |
| rs4415084 | 44698272 | rs2218081 | 44740897 | 1.000 | 1.0 | 47.37 |
| rs4415084 | 44698272 | rs16901937 | 44744898 | 1.000 | 0.978 | 45.06 |
| rs4415084 | 44698272 | rs11747159 | 44773467 | 0.948 | 0.747 | 28.79 |
| rs4415084 | 44698272 | rs2330572 | 44776746 | 0.952 | 0.845 | 34.31 |
| rs4415084 | 44698272 | rs994793 | 44779004 | 0.952 | 0.848 | 34.49 |
| rs4415084 | 44698272 | rs1438827 | 44787713 | 0.948 | 0.749 | 29.76 |
| rs4415084 | 44698272 | rs7712949 | 44806102 | 0.948 | 0.746 | 29.19 |
| rs4415084 | 44698272 | rs11746980 | 44813635 | 0.952 | 0.848 | 34.49 |
| rs4415084 | 44698272 | rs16901964 | 44819012 | 0.949 | 0.768 | 30.54 |
| rs4415084 | 44698272 | rs727305 | 44831799 | 0.972 | 0.746 | 27.65 |
| rs4415084 | 44698272 | rs10462081 | 44836422 | 0.948 | 0.749 | 29.76 |
| rs4415084 | 44698272 | rs13183209 | 44839506 | 0.925 | 0.746 | 28.55 |
| rs4415084 | 44698272 | rs13159598 | 44841683 | 0.952 | 0.848 | 34.19 |
| rs4415084 | 44698272 | rs3761650 | 44844113 | 0.947 | 0.744 | 28.68 |
| rs4415084 | 44698272 | rs13174122 | 44846497 | 0.971 | 0.735 | 26.70 |
| rs4415084 | 44698272 | rs11746506 | 44848323 | 0.973 | 0.764 | 29.24 |
| rs4415084 | 44698272, | rs7720787 | 44853066 | 0.952 | 0.845 | 34.31 |
| rs4415084 | 44698272 | rs9637783 | 44855403 | 0.948 | 0.748 | 29.16 |
| rs4415084 | 44698272 | rs4457089 | 44857493 | 0.948 | 0.762 | 29.70 |
| rs4415084 | 44698272 | rs6896350 | 44868328 | 0.948 | 0.764 | 29.46 |
| rs4415084 | 44698272 | rs1371025 | 44869990 | 0.973 | 0.785 | 30.69 |
| rs4415084 | 44698272 | rs4596389 | 44872313 | 0.948 | 0.749 | 29.76 |
| rs4415084 | 44698272 | rs6451775 | 44872545 | 0.948 | 0.746 | 29.19 |
| rs4415084 | 44698272 | rs729599 | 44878017 | 0.948 | 0.748 | 29.16 |
| rs4415084 | 44698272 | rs987394 | 44882135 | 0.948 | 0.749 | 29.76 |
| rs4415084 | 44698272 | rs4440370 | 44889109 | 0.948 | 0.748 | 29.16 |
| rs4415084 | 44698272 | rs7703497 | 44892785 | 0.948 | 0.749 | 29.76 |
| rs4415084 | 44698272 | rs13362132 | 44894017 | 0.952 | 0.827 | 34.09 |
| rs4415084 | 44698272 | rs1438821 | 44894208 | 0.951 | 0.844 | 34.52 |

TABLE 4
Surrogate markers for SNP rs13387042. Markers with a r2
greater than 0.05 to rs13387042 in the HAPMAP dataset
(http://hapmap.ncbi.nlm.nih.gov) in a 1 Mbp interval
flanking the marker was selected. Shown is the name of the
correlated SNP, values for r2 and D′ to rs13387042 and the
corresponding LOD value, as well as the position of the
surrogate marker in NCB Build 36.

| DbSNP | | Correlated | | | | |
|---|---|---|---|---|---|---|
| rsID | Position | SNP | Location | D′ | r² | LOD |
| rs13387042 | 217614077 | rs4621152 | 217617230 | 0.865 | 0.364 | 15.30 |
| rs13387042 | 217614077 | rs6721996 | 217617708 | 1.000 | 0.979 | 50.46 |
| rs13387042 | 217614077 | rs12694403 | 217623659 | 0.955 | 0.33 | 14.24 |
| rs13387042 | 217614077 | rs17778427 | 217631258 | 1.000 | 0.351 | 16.12 |
| rs13387042 | 217614077 | rs17835044 | 217631850 | 1.000 | 0.351 | 16.12 |
| rs13387042 | 217614077 | rs7588345 | 217632061 | 1.000 | 0.193 | 8.93 |
| rs13387042 | 217614077 | rs7562029 | 217632506 | 1.000 | 0.413 | 20.33 |
| rs13387042 | 217614077 | rs13000023 | 217632639 | 0.949 | 0.287 | 12.20 |
| rs13387042 | 217614077 | rs13409592 | 217634573 | 0.933 | 0.192 | 7.69 |
| rs13387042 | 217614077 | rs2372957 | 217635302 | 0.855 | 0.168 | 5.97 |
| rs13387042 | 217614077 | rs16856888 | 217638914 | 0.363 | 0.101 | 3.31 |
| rs13387042 | 217614077 | rs16856890 | 217639976 | 0.371 | 0.101 | 3.29 |
| rs13387042 | 217614077 | rs7598926 | 217640464 | 0.382 | 0.109 | 3.60 |
| rs13387042 | 217614077 | rs6734010 | 217643676 | 0.543 | 0.217 | 7.90 |
| rs13387042 | 217614077 | rs13022815 | 217644369 | 0.800 | 0.319 | 12.94 |
| rs13387042 | 217614077 | rs16856893 | 217645298 | 0.739 | 0.109 | 3.45 |
| rs13387042 | 217614077 | rs13011060 | 217646422 | 0.956 | 0.352 | 14.71 |
| rs13387042 | 217614077 | rs4674132 | 217646764 | 0.802 | 0.327 | 13.10 |
| rs13387042 | 217614077 | rs16825211 | 217647249 | 0.912 | 0.326 | 12.95 |
| rs13387042 | 217614077 | rs41521045 | 217647581 | 0.903 | 0.112 | 4.70 |
| rs13387042 | 217614077 | rs2372960 | 217650960 | 0.678 | 0.058 | 2.12 |
| rs13387042 | 217614077 | rs2372967 | 217676158 | 0.326 | 0.052 | 1.97 |
| rs13387042 | 217614077 | rs3843337 | 217677680 | 0.326 | 0.052 | 1.97 |
| rs13387042 | 217614077 | rs2372972 | 217679386 | 0.375 | 0.062 | 2.28 |
| rs13387042 | 217614077 | rs9677455 | 217680497 | 0.375 | 0.062 | 2.28 |
| rs13387042 | 217614077 | rs12464728 | 217686802 | 0.478 | 0.073 | 2.54 |

In another embodiment, when determining breast cancer risk, the methods of the present disclosure encompass assessing all of the SNPs shown in Table 6 or a single nucleotide polymorphism in linkage disequilibrium with one or more thereof.

Table 6 and Table 7 recite overlapping SNPs. It will be appreciated that when selecting SNPs for assessment the same SNP will not be selected twice. For convenience, the SNPs in Table 6 have been separated into Tables 7 and 8. Table 7 lists SNPs common across Caucasians, African American and Hispanic populations. Table 8 lists SNPs that are not common across Caucasians, African American and Hispanic populations.

In a further embodiment, between 72 and 88, between 73 and 87, between 74 and 86, between 75 and 85, between 76 and 84, between 75 and 83, between 76 and 82, between 77 and 81, between 78 and 80 single nucleotide polymorphisms are assessed, wherein at least 60, at least 61, at least 62, at least 63, at least 64, at least 65, at least 66, at least 67, at least 68, at least 69, at least 70, of the SNPs shown in Table 7, or a single nucleotide polymorphism in linkage disequilibrium with one or more thereof are assessed, with any remaining SNPs being selected from Table 6, or a single nucleotide polymorphism in linkage disequilibrium with one or more thereof.

In an embodiment, the number of SNPs assessed is based on the net reclassification improvement in risk prediction calculated using net reclassification index (NRI) (Pencina et al., 2008).

In an embodiment, the net reclassification improvement of the methods of the present disclosure is greater than 0.01.

In a further embodiment, the net reclassification improvement of the methods of the present disclosure is greater than 0.05.

In yet another embodiment, the net reclassification improvement of the methods of the present disclosure is greater than 0.1.

In another embodiment the genetic risk assessment is performed by analysing the genotype of the subject at 90 or more loci for single nucleotide polymorphisms associated with breast cancer. In another embodiment, the genetic risk assessment is performed by analysing the genotype of the subject at 100, 200, 300, 400, 500, 600, 700, 800, 900, 1,000, 5,000, 10,000, 50,000, 100,000 or more loci for single nucleotide polymorphisms associated with breast cancer. In these embodiments, one or more of the SNPs can be selected from Tables 6 to 11.

### Ethnic Genotype Variation

It is known to those of skill in the art that genotypic variation exists between different populations. This phenomenon is referred to as human genetic variation. Human genetic variation is often observed between populations from different ethnic backgrounds. Such variation is rarely consistent and is often directed by various combinations of environmental and lifestyle factors. As a result of genetic variation, it is often difficult to identify a population of genetic markers such as SNPs that remain informative across various populations such as populations from different ethnic backgrounds.

A selection of SNPs that are common to at least three ethnic backgrounds and remain informative for assessing the risk for developing breast cancer are disclosed herein.

In an embodiment, the methods of the present disclosure can be used for assessing the risk for developing breast cancer in human female subjects from various ethnic backgrounds. For example, the female subject can be classified as Caucasoid, Australoid, Mongoloid and Negroid based on physical anthropology.

In an embodiment, the human female subject can be Caucasian, African American, Hispanic, Asian, Indian, or Latino. In a preferred embodiment, the human female subject is Caucasian, African American or Hispanic. Accordingly, ethnicity can be taken into consideration as part of the clinical and/or genetic risk assessments.

In one embodiment, the human female subject is Caucasian and at least 72, at least 73, at least 74, at least 75, at least 76, at least 77, single nucleotide polymorphisms selected from Table 9, or a single nucleotide polymorphism in linkage disequilibrium therewith are assessed. Alternatively, at least 77 single nucleotide polymorphisms selected from Table 9 or a single nucleotide polymorphism in linkage disequilibrium therewith are assessed.

In another embodiment, the human female subject can be Negroid and at least 74, at least 75, at least 76, at least 77, at least 78, single nucleotide polymorphisms selected from Table 10, or a single nucleotide polymorphism in linkage disequilibrium therewith are assessed. Alternatively, at least 78 single nucleotide polymorphisms selected from Table 10 or a single nucleotide polymorphism in linkage disequilibrium therewith are assessed.

In another embodiment, the human female subject can be African American and at least 74, at least 75, at least 76, at least 77, at least 78, single nucleotide polymorphisms selected from Table 10, or a single nucleotide polymorphism in linkage disequilibrium therewith are assessed. Alternatively, at least 78 single nucleotide polymorphisms selected from Table 10 or a single nucleotide polymorphism in linkage disequilibrium therewith are assessed.

In a further embodiment, the human female subject can be Hispanic and at least 78, at least 79, at least 80, at least 81, at least 82, single nucleotide polymorphisms selected from Table 11, or a single nucleotide polymorphism in linkage disequilibrium therewith are assessed. Alternatively, at least 82 single nucleotide polymorphisms selected from Table 11 or a single nucleotide polymorphism in linkage disequilibrium therewith are assessed.

It is well known that over time there has been blending of different ethnic origins. However, in practice this does not influence the ability of a skilled person to practice the invention.

A female subject of predominantly European origin, either direct or indirect through ancestry, with white skin is considered Caucasian in the context of the present disclosure. A Caucasian may have, for example, at least 75% Caucasian ancestry (for example, but not limited to, the female subject having at least three Caucasian grandparents).

A female subject of predominantly central or southern African origin, either direct or indirect through ancestry, is considered Negroid in the context of the present disclosure. A Negroid may have, for example, at least 75% Negroid ancestry. An American female subject with predominantly Negroid ancestry and black skin is considered African American in the context of the present disclosure. An African American may have, for example, at least 75% Negroid ancestry. Similar principle applies to, for example, females of Negroid ancestry living in other countries (for example Great Britain, Canada and The Netherlands).

A female subject predominantly originating from Spain or a Spanish-speaking country, such as a country of Central or Southern America, either direct or indirect through ancestry, is considered Hispanic in the context of the present disclosure. An Hispanic may have, for example, at least 75% Hispanic ancestry.

The terms “ethnicity” and “race” can be used interchangeably in the context of the present disclosure. In an embodiment, the genetic risk assessment can readily be practiced based on what ethnicity the subject considers themselves to be. Thus, in an embodiment, the ethnicity of the human female subject is self-reported by the subject. As an example, female subjects can be asked to identify their ethnicity in response to this question: “To what ethnic group do you belong?”. In another example, the ethnicity of the female subject is derived from medical records after obtaining the appropriate consent from the subject or from the opinion or observations of a clinician.

### Calculating Composite SNP Relative Risk “SNP Risk”

An individual's composite SNP relative risk score (“SNP risk”) can be defined as the product of genotype relative risk values for each SNPs assessed. A log-additive risk model can then be used to define three genotypes AA, AB, and BB for a single SNP having relative risk values of 1, OR, and OR², under a rare disease model, where OR is the previously reported disease odds ratio for the high-risk allele, B, vs the low-risk allele, A. If the B allele has frequency (p), then these genotypes have population frequencies of (1−p)², 2p(1−p), and p², assuming Hardy-Weinberg equilibrium. The genotype relative risk values for each SNP can then be scaled so that based on these frequencies the average relative risk in the population is 1. Specifically, given the unscaled population average relative risk:

(μ)=(1−p)²+2p(1−p)OR+p²OR²

Adjusted risk values 1/μ, OR/μ, and OR²/μ are used for AA, AB, and BB genotypes. Missing genotypes are assigned a relative risk of 1.

Similar calculations can be performed for non-SNP polymorphisms.

### Combined Clinical Risk×Genetic Risk

It is envisaged that the “risk” of a human female subject for developing breast cancer can be provided as a relative risk (or risk ratio) or an absolute risk as required. In an embodiment, the clinical risk assessment is combined with the genetic risk assessment to obtain the “absolute risk” of a human female subject for developing breast cancer.

Absolute risk is the numerical probability of a human female subject developing breast cancer within a specified period (e.g. 5, 10, 15, 20 or more years). It reflects a human female subjects risk of developing breast cancer in so far as it does not consider various risk factors in isolation.

One example of combining the clinical risk assessment with the genetic risk assessment to obtain the “absolute risk” of a human female subject for developing breast cancer involves using the following formula:

abs_risk=mortsuv(1−exp(−RR×SNP(incid_5−incid_age)))
Where RR=the relative risk associated with having a first degree relative with breast cancer, SNP is the composite SNP relative risk determined by genetic risk assessment, incid_age is the breast cancer incidence at the current (baseline) age, incid_5 is the breast cancer incidence at baseline+5 years, mortsurv is the competing mortality due to causes other than breast cancer.

Breast cancer incidence and competing mortality data can be obtained from various sources. In, an example these data are obtained from the United States Surveillance, Epidemiology, and End Results Program (SEER) database.

In an embodiment, ethnic-specific breast cancer incidence and competing mortality data are used in the above formula. In an example, ethnic-specific breast cancer incidence and competing mortality data can also be obtained from the SEER database.

Various suitable databases can be used to calculate the relative risk associated with a female subjects family history of breast cancer. One example is provided by the Cancer, Collaborative Group on Hormonal Factors in Breast Cancer (CGoHFiB). In another example, relevant population statistics can be obtained from the Seer database (Siegel et al. 2016).

In another embodiment, the clinical risk assessment is combined with the genetic risk assessment to obtain the “relative risk” of a human female subject for developing breast cancer. Relative risk (or risk ratio), measured as the incidence of a disease in individuals with a particular characteristic (or exposure) divided by the incidence of the disease in individuals without the characteristic, indicates whether that particular exposure increases or decreases risk. Relative risk is helpful to identify characteristics that are associated with a disease, but by itself is not particularly helpful in guiding screening decisions because the frequency of the risk (incidence) is cancelled out.

In combining the clinical risk assessment with the genetic risk assessment to obtain the “risk” of a human female subject for developing breast cancer, the following formula can be used:

[Risk (i.e. Clinical Evaluation×SNP risk)]=[Clinical Evaluation risk]×SNP₁×SNP₂×SNP₃×SNP₄×SNP₅×SNP₆×SNP₇,×SNP×, . . . etc.

Where Clinical Evaluation is the risk score provided by the clinical evaluation, and SNP₁ to SNPₓ are relative risk scores for the individual SNPs, each scaled to have a population average of 1 as outlined above. Because the SNP risk scores have been “centred” to have a population average risk of 1, if one assumes independence among the SNPs, then the population average risk across all genotypes for the combined score is consistent with the underlying Clinical Evaluation risk estimate.

In an embodiment, the risk of a human female subject for developing breast cancer is calculated by [5-year age risk score]×[5-year family history of breast cancer among first degree relatives risk score]×SNP₁×SNP₂×SNP₃×SNP₄×SNP₅×SNP₆×SNP₇×SNPₓ, . . . etc.

In another embodiment, the risk of a human female subject for developing breast cancer is calculated by [lifetime age risk score]×[lifetime family history of breast cancer among first degree relatives risk score]×SNP₁×SNP₂×SNP₃×SNP₄×SNP₅×SNP₆×SNP₇×SNPₓ, . . . etc.

In an embodiment, the risk [Clinical 5-year risk×SNP risk] is used to determine whether a chemopreventative should be offered to a subject to reduce the subjects risk. For example, the risk [Clinical 5-year risk×SNP risk] can be used to determine whether estrogen receptor therapy should be offered to a subject to reduce the subjects risk. In this embodiment, the threshold level of risk is preferably >1.66% for 5-year risk.

In a further embodiment, the risk [Clinical lifetime risk×SNP risk] is used to determine whether a subject should be enrolled screening breast MRI and mammography program. In this embodiment, the threshold level is preferably greater than about (20% lifetime risk).

### Treatment

After performing the methods of the present disclosure treatment may be prescribed or administered to the subject.

Accordingly, in an embodiment, the methods of the present disclosure relate to an anti-cancer therapy for use in preventing or reducing the risk of breast cancer in a human subject at risk thereof.

One of skill in the art will appreciate that breast cancer is a heterogeneous disease with distinct clinical outcomes (Sorlie et al., 2001). For example, it is discussed in the art that breast cancer may be estrogen receptor positive or estrogen receptor negative. In one embodiment, it is not envisaged that the methods of the present disclosure be limited to assessing the risk of developing a particular type or subtype of breast cancer. For example, it is envisaged that the methods of the present disclosure can be used to assess the risk of developing estrogen receptor positive or estrogen receptor negative breast cancer. In another embodiment, the methods of the present disclosure are used to assess the risk of developing estrogen receptor positive breast cancer. In another embodiment, the methods of the present disclosure are used to assess the risk of developing estrogen receptor negative breast cancer. In another embodiment, the methods of the present disclosure are used to assess the risk of developing metastatic breast cancer. In an example, a therapy that inhibits estrogen is prescribed or administered to the subject.

In another example, a chemopreventative is prescribed or administered to the subject. There are two main classes of drugs currently utilized for breast cancer chemoprevention:
(1) Selective Estrogen Receptor Modulators (SERMs) which block estrogen molecules from binding to their associated cellular receptor. This class of drugs includes for example Tamoxifen and Raloxifene.
(2) Aromatase Inhibitors which inhibit the conversion of androgens into estrogens by the aromatase enzyme Ie reducing the production of estrogens. This class of drugs includes for example Exemestane, Letrozole, Anastrozole, Vorozole, Formestane, Fadrozole.

In an example, a SERM or an aromatase inhibitor is prescribed or administered to the subject.

In an example, Tamoxifen, Raloxifene, Exemestane, Letrozole, Anastrozole, Vorozole, Formestane or Fadrozole is prescribed or administered to a subject.

In an embodiment, the methods of the present disclosure are used to assess the risk of a human female subject for developing breast cancer and administering a treatment appropriate for the risk of developing breast cancer. For example, when performing the methods of the present disclosure indicates a high risk of breast cancer an aggressive chemopreventative treatment regimen can be established. In contrast, when performing the methods of the present disclosure indicates a moderate risk of breast cancer a less aggressive chemopreventative treatment regimen can be established.

Alternatively, when performing the methods of the present disclosure indicates a low risk of breast cancer a chemopreventative treatment regimen need not be established. It is envisaged that the methods of the present disclosure can be performed over time so that the treatment regimen can be modified in accordance with the subjects risk of developing breast cancer.

### Marker Detection Strategies

Amplification primers for amplifying markers (e.g., marker loci) and suitable probes to detect such markers or to genotype a sample with respect to multiple marker alleles, can be used in the disclosure. For example, primer selection for long-range PCR is described in U.S. Ser. No. 10/042,406 and U.S. Ser. No. 10/236,480; for short-range PCR, U.S. Ser. No. 10/341,832 provides guidance with respect to primer selection. Also, there are publicly available programs such as “Oligo” available for primer design. With such available primer selection and design software, the publicly available human genome sequence and the polymorphism locations, one of skill can construct primers to amplify the SNPs to practice the disclosure. Further, it will be appreciated that the precise probe to be used for detection of a nucleic acid comprising a SNP (e.g., an amplicon comprising the SNP) can vary, e.g., any probe that can identify the region of a marker amplicon to be detected can be used in conjunction with the present disclosure. Further, the configuration of the detection probes can, of course, vary. Thus, the disclosure is not limited to the sequences recited herein.

Indeed, it will be appreciated that amplification is not a requirement for marker detection, for example one can directly detect unamplified genomic DNA simply by performing a Southern blot on a sample of genomic DNA.

Typically, molecular markers are detected by any established method available in the art, including, without limitation, allele specific hybridization (ASH), detection of single nucleotide extension, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, and single-strand conformation polymorphisms (SSCP) detection.

Examples of oligonucleotide primers useful for amplifying nucleic acids comprising SNPs associated with breast cancer are provided in Table 5. As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

In some embodiments, the primers of the disclosure are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

TABLE 5
Examples of oligonucleotide primers
useful for the disclosure.

| Name | Sequence |
|---|---|
| rs889312_for | TATGGGAAGGAGTCGTTGAG |
| | (SEQ ID NO: 1) |
| rs6504950_for | CTGAATCACTCCTTGCCAAC |
| | (SEQ ID NO: 2) |
| rs4973768_for | CAAAATGATCTGACTACTCC |
| | (SEQ ID NO: 3) |
| rs4415084_for | TGACCAGTGCTGTATGTATC |
| | (SEQ ID NO: 4) |
| rs3817198_for | TCTCACCTGATACCAGATTC |
| | (SEQ ID NO: 5) |
| rs3803662_for | TCTCTCCTTAATGCCTCTAT |
| | (SEQ ID NO: 6) |
| rs2981582_for | ACTGCTGCGGGTTCCTAAAG |
| | (SEQ ID NO: 7) |
| rs13387042_for | GGAAGATTCGATTCAACAAGG |
| | (SEQ ID NO: 8) |
| rs13281615_for | GGTAACTATGAATCTCATC |
| | (SEQ ID NO: 9) |
| rs11249433_for | AAAAAGCAGAGAAAGCAGGG |
| | (SEQ ID NO: 10) |
| rs889312_rev | AGATGATCTCTGAGATGCCC |
| | (SEQ ID NO: 11) |
| rs6504950_rev | CCAGGGTTTGTCTACCAAAG |
| | (SEQ ID NO: 12) |
| rs4973768_rev | AATCACTTAAAACAAGCAG |
| | (SEQ ID NO: 13) |
| rs4415084_rev | CACATACCTCTACCTCTAGC |
| | (SEQ ID NO: 14) |
| rs3817198_rev | TTCCCTAGTGGAGCAGTGG |
| | (SEQ ID NO: 15) |
| rs3803662_rev | CTTTCTTCGCAAATGGGTGG |
| | (SEQ ID NO: 16) |
| rs2981582_rev | GCACTCATCGCCACTTAATG |
| | (SEQ ID NO: 17) |
| rs13387042_rev | GAACAGCTAAACCAGAACAG |
| | (SEQ ID NO: 18) |
| rs13281615_rev | ATCACTCTTATTTCTCCCCC |
| | (SEQ ID NO: 19) |
| rs11249433_rev | TGAGTCACTGTGCTAAGGAG |
| | (SEQ ID NO: 20) |

It is not intended that the primers of the disclosure be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook et al. (supra).

PCR detection using dual-labelled fluorogenic oligonucleotide probes, commonly referred to as “TaqMan™” probes, can also be performed according to the present disclosure. These probes are composed of short (e.g., 20-25 base) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the “LightCycler®” format described in U.S. Pat. No. 6,174,670.

Array-based detection can be performed using commercially available arrays, e.g., from Affymetrix (Santa Clara, Calif.) or other manufacturers. Reviews regarding the operation of nucleic acid arrays include Sapolsky et al. (1999); Lockhart (1998); Fodor (1997a); Fodor (1997b) and Chee et al. (1996). Array based detection is one preferred method for identification markers of the disclosure in samples, due to the inherently high-throughput nature of array based detection.

The nucleic acid sample to be analysed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

### Correlating Markers to Phenotypes

These correlations can be performed by any method that can identify a relationship between an allele and a phenotype, or a combination of alleles and a combination of phenotypes. For example, alleles in genes or loci defined herein can be correlated with one or more breast cancer phenotypes. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present disclosure (Hartl et al., 1981). A variety of appropriate statistical models are described in Lynch and Walsh (1998). These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as the use of genetic algorithms, can be used to determine correlations between markers and phenotypes. This is particularly useful when identifying higher order correlations between multiple alleles and multiple phenotypes. To illustrate, neural network approaches can be coupled to genetic algorithm-type programming for heuristic development of a structure-function data space model that determines correlations between genetic information and phenotypic outcomes.

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www.partek.com), e.g., that provide software for pattern recognition (e.g., which provide Partek Pro 2000 Pattern Recognition Software).

Additional details regarding association studies can be found in U.S. Ser. No. 10/106,097, U.S. Ser. No. 10/042,819, U.S. Ser. No. 10/286,417, U.S. Ser. No. 10/768,788, U.S. Ser. No. 10/447,685, U.S. Ser. No. 10/970,761, and U.S. Pat. No. 7,127,355.

Systems for performing the above correlations are also a feature of the disclosure. Typically, the system will include system instructions that correlate the presence or absence of an allele (whether detected directly or, e.g., through expression levels) with a predicted phenotype.

Optionally, the system instructions can also include software that accepts diagnostic information associated with any detected allele information, e.g., a diagnosis that a subject with the relevant allele has a particular phenotype. This software can be heuristic in nature, using such inputted associations to improve the accuracy of the look up tables and/or interpretation of the look up tables by the system. A variety of such approaches, including neural networks, Markov modelling, and other statistical analysis are described above.

### Polymorphic Profiling

The disclosure provides methods of determining the polymorphic profile of an individual at the SNPs outlined in the present disclosure (e.g. Table 6) or SNPs in linkage disequilibrium with one or more thereof.

The polymorphic profile constitutes the polymorphic forms occupying the various polymorphic sites in an individual. In a diploid genome, two polymorphic forms, the same or different from each other, usually occupy each polymorphic site. Thus, the polymorphic profile at sites X and Y can be represented in the form X (x1, x1), and Y (y1, y2), wherein x1, x1 represents two copies of allele x1 occupying site X and y1, y2 represent heterozygous alleles occupying site Y.

The polymorphic profile of an individual can be scored by comparison with the polymorphic forms associated with resistance or susceptibility to breast cancer occurring at each site. The comparison can be performed on at least, e.g., 1, 2, 5, 10, 25, 50, or all of the polymorphic sites, and optionally, others in linkage disequilibrium with them. The polymorphic sites can be analysed in combination with other polymorphic sites.

Polymorphic profiling is useful, for example, in selecting agents to affect treatment or prophylaxis of breast cancer in a given individual. Individuals having similar polymorphic profiles are likely to respond to agents in a similar way.

Polymorphic profiling is also useful for stratifying individuals in clinical trials of agents being tested for capacity to treat breast cancer or related conditions. Such trials are performed on treated or control populations having similar or identical polymorphic profiles (see EP 99965095.5), for example, a polymorphic profile indicating an individual has an increased risk of developing breast cancer. Use of genetically matched populations eliminates or reduces variation in treatment outcome due to genetic factors, leading to a more accurate assessment of the efficacy of a potential drug.

Polymorphic profiling is also useful for excluding individuals with no predisposition to breast cancer from clinical trials. Including such individuals in the trial increases the size of the population needed to achieve a statistically significant result. Individuals with no predisposition to breast cancer can be identified by determining the numbers of resistances and susceptibility alleles in a polymorphic profile as described above. For example, if a subject is genotyped at ten sites in ten genes of the disclosure associated with breast cancer, twenty alleles are determined in total. If over 50% and alternatively over 60% or 75% percent of these are resistance genes, the individual is unlikely to develop breast cancer and can be excluded from the trial.

In other embodiments, stratifying individuals in clinical trials may be accomplished using polymorphic profiling in combination with other stratification methods, including, but not limited to risk models (e.g., Gail Score, Claus model), clinical phenotypes (e.g., atypical lesions, breast density), and specific candidate biomarkers.

### Computer Implemented Method

It is envisaged that the methods of the present disclosure may be implemented by a system such as a computer implemented method. For example, the system may be a computer system comprising one or a plurality of processors which may operate together (referred to for convenience as “processor”) connected to a memory. The memory may be a non-transitory computer readable medium, such as a hard drive, a solid state disk or CD-ROM. Software, that is executable instructions or program code, such as program code grouped into code modules, may be stored on the memory, and may, when executed by the processor, cause the computer system to perform functions such as determining that a task is to be performed to assist a user to determine the risk of a human female subject for developing breast cancer; receiving data indicating the clinical risk and genetic risk of the female subject developing breast cancer, wherein the genetic risk was derived by detecting, in a biological sample derived from the female subject, at least 72 single nucleotide polymorphisms associated with breast cancer, wherein at least 67 of the single nucleotide polymorphisms are selected from Table 7, or a single nucleotide polymorphism in linkage disequilibrium with one or more thereof and the remaining single nucleotide polymorphisms are selected from Table 6, or a single nucleotide polymorphism in linkage disequilibrium with one or more thereof; processing the data to combine the clinical risk with the genetic risk assessment to obtain the risk of a human female subject for developing breast cancer; outputting the risk of a human female subject for developing breast cancer.

For example, the memory may comprise program code which when executed by the processor causes the system to determine at least 72 single nucleotide polymorphisms associated with breast cancer, wherein at least 67 of the single nucleotide polymorphisms are selected from Table 7, or a single nucleotide polymorphism in linkage disequilibrium with one or more thereof and the remaining single nucleotide polymorphisms are selected from Table 6, or a single nucleotide polymorphism in linkage disequilibrium with one or more thereof, or receive data indicating at least 72 single nucleotide polymorphisms associated with breast cancer, wherein at least 67 of the single nucleotide polymorphisms are selected from Table 7, or a single nucleotide polymorphism in linkage disequilibrium with one or more thereof and the remaining single nucleotide polymorphisms are selected from Table 6, or a single nucleotide polymorphism in linkage disequilibrium with one or more thereof; process the data to combine the clinical risk with the genetic risk assessment to obtain the risk of a human female subject for developing breast cancer; report the risk of a human female subject for developing breast cancer.

In another embodiment, the system may be coupled to a user interface to enable the system to receive information from a user and/or to output or display information. For example, the user interface may comprise a graphical user interface, a voice user interface or a touchscreen.

In an embodiment, the program code may causes the system to determine the “SNP risk”.

In an embodiment, the program code may causes the system to determine Combined Clinical assessment×Genetic Risk (for example SNP risk).

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, performing the methods of the present disclosure to assess the risk of a human female subject for developing breast cancer, enables establishment of a diagnostic or prognostic rule based on the clinical risk and genetic risk of the female subject developing breast cancer. For example, the diagnostic or prognostic rule can be based on the Combined Clinical assessment×SNP Risk Score relative to a control, standard or threshold level of risk.

In an embodiment, the threshold level of risk is the level recommended by the American Cancer Society (ACS) guidelines for screening breast MRI and mammography. In this example, the threshold level is preferably greater than about (20% lifetime risk).

In another embodiment, the threshold level of risk is the level recommended American Society of Clinical Oncology (ASCO) for offering an estrogen receptor therapy to reduce a subjects risk. In this embodiment, the threshold level of risk is preferably (GAIL index>1.66% for 5-year risk).

In another embodiment, the diagnostic or prognostic rule is based on the application of a statistical and machine learning algorithm. Such an algorithm uses relationships between a population of SNPs and disease status observed in training data (with known disease status) to infer relationships which are then used to determine the risk of a human female subject for developing breast cancer in subjects with an unknown risk. An algorithm is employed which provides an risk of a human female subject developing breast cancer. The algorithm performs a multivariate or univariate analysis function.

### Single Nucleotide Polymorphisms Indicative of Breast Cancer Risk

Examples of SNPs indicative of breast cancer risk are shown in Table 6. 77 SNPs are informative in Caucasians, 78 SNPs are informative in African Americans and 82 are informative in Hispanics. 70 SNPs are informative in Caucasians, African Americans and Hispanics (indicated by horizontal stripe pattern; see also Table 7). The remaining 18 SNPs (see Table 8) are informative in either Caucasians (indicated by dark trellis pattern; see also Table 9), African Americans (indicated by downward diagonal stripe pattern; see also Table 10) and/or Hispanics (indicated by light grid pattern; see also Table 11).

TABLE 9
Caucasian SNPs (n = 77). Alleles represented as major/minor (eg for
rs616488 A is the common allele and G less common). OR minor allele
numbers below 1 means the minor allele is not the risk allele,
whereas when above 1 the minor allele is the risk allele.

| | | | Minor | OR | | | | | | | |
|---|---|---|---|---|---|---|---|---|---|---|---|
| | | | allele | Minor | | | | | | | |
| SNP | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.0661 | 1.05 | AA | 0,95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | CC | 1.07 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rS132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

TABLE 10
African American SNPs (n = 78). Alleles represented as risk/
reference (non risk) (eg for rs616488 A is the risk allele).

| | | | Risk | OR | | | | | | | |
|---|---|---|---|---|---|---|---|---|---|---|---|
| | | | allele | Risk | | | | | | | |
| SNP | Chromosome | Alleles | frequency | Allele | μ | Adjusted Risk Score | | | | | |
| rs616488 | 1 | A/G | 0.86 | 1.03 | 1.05 | AA | 0.95 | AG | 0.98 | GG | 1.01 |
| rs11552449 | 1 | C/T | 0.037 | 0.9 | 0.99 | CC | 1.01 | CT | 0.91 | TT | 0.82 |
| rs11249433 | 1 | A/G | 0.13 | 0.99 | 1.00 | AA | 1.00 | AG | 0.99 | GG | 0.98 |
| rs6678914 | 1 | G/A | 0.66 | 1 | 1.00 | GG | 1.00 | GA | 1.00 | AA | 1.00 |
| rs4245739 | 1 | A/C | 0.24 | 0.97 | 0.99 | AA | 1.01 | AC | 0.98 | CC | 0.95 |
| rs12710696 | 2 | G/A | 0.53 | 1.06 | 1.06 | GG | 0.94 | GA | 1.00 | AA | 1.06 |
| rs4849887 | 2 | C/T | 0.7 | 1.16 | 1.24 | CC | 0.81 | CT | 0.94 | TT | 1.09 |
| rs2016394 | 2 | G/A | 0.72 | 1.05 | 1.07 | GG | 0.93 | GA | 0.98 | AA | 1.03 |
| rs1550623 | 2 | A/G | 0.71 | 1.1 | 1.15 | AA | 0.87 | AG | 0.96 | GG | 1.05 |
| rs1045485 | 2 | G/C | 0.93 | 0.99 | 0.98 | GG | 1.02 | GC | 1.01 | CC | 1.00 |
| rs13387042 | 2 | A/G | 0.72 | 1.12 | 1.18 | AA | 0.85 | AG | 0.95 | GG | 1.06 |
| rs16857609 | 2 | C/T | 0.24 | 1.17 | 1.08 | CC | 0.92 | CT | 1.08 | TT | 1.26 |
| rs6762644 | 3 | A/G | 0.46 | 1.05 | 1.05 | AA | 0.96 | AG | 1.00 | GG | 1.05 |
| rs4973768 | 3 | C/T | 0.36 | 1.04 | 1.03 | CC | 0.97 | CT | 1.01 | TT | 1.05 |
| rs12493607 | 3 | G/C | 0.14 | 1.04 | 1.01 | GG | 0.99 | GC | 1.03 | CC | 1.07 |
| rs9790517 | 4 | C/T | 0.084 | 0.88 | 0.98 | CC | 1.02 | CT | 0.90 | TT | 0.79 |
| rs6828523 | 4 | C/A | 0.65 | 1 | 1.00 | CC | 1.00 | CA | 1.00 | AA | 1.00 |
| rs4415084 | 5 | C/T | 0.61 | 1.1 | 1.13 | CC | 0.89 | CT | 0.98 | TT | 1.07 |
| rs10069690 | 5 | C/T | 0.57 | 1.13 | 1.15 | CC | 0.87 | CT | 0.98 | TT | 1.11 |
| rs10941679 | 5 | A/G | 0.21 | 1.04 | 1.02 | AA | 0.98 | AG | 1.02 | GG | 1.06 |
| rs889312 | 5 | A/C | 0.33 | 1.07 | 1.05 | AA | 0.96 | AC | 1.02 | CC | 1.09 |
| rs10472076 | 5 | T/C | 0.28 | 0.95 | 0.97 | TT | 1.03 | TC | 0.98 | CC | 0.93 |
| rs1353747 | 5 | T/G | 0.98 | 1.01 | 1.02 | TT | 0.98 | TG | 0.99 | GG | 1.00 |
| rs1432679 | 5 | A/G | 0.79 | 1.07 | 1.11 | AA | 0.90 | AG | 0.96 | GG | 1.03 |
| rs11242675 | 6 | T/C | 0.51 | 1.06 | 1.06 | TT | 0.94 | TC | 1.00 | CC | 1.06 |
| rs204247 | 6 | T/C | 0.34 | 1.13 | 1.09 | AA | 0.92 | AG | 1.04 | GG | 1.17 |
| rs17529111 | 6 | A/G | 0.075 | 0.99 | 1.00 | AA | 1.00 | AG | 0.99 | GG | 0.98 |
| rs9485370 | 6 | G/T | 0.78 | 1.13 | 1.21 | GG | 0.82 | GT | 0.93 | TT | 1.05 |
| rs3757318 | 6 | G/A | 0.038 | 1.11 | 1.01 | GG | 0.99 | GA | 1.10 | AA | 1.22 |
| rs2046210 | 6 | G/A | 0.6 | 0.99 | 0.99 | GG | 1.01 | GA | 1.00 | AA | 0.99 |
| rs720475 | 7 | G/A | 0.88 | 0.99 | 0.98 | GG | 1.02 | GA | 1.01 | AA | 1.00 |
| rs9693444 | 8 | C/A | 0.37 | 1.06 | 1.04 | CC | 0.96 | CA | 1.01 | AA | 1.08 |
| rs6472903 | 8 | T/G | 0.9 | 1.02 | 1.04 | TT | 0.96 | TG | 0.98 | GG | 1.00 |
| rs2943559 | 8 | A/G | 0.22 | 1.07 | 1.03 | AA | 0.97 | AG | 1.04 | GG | 1.11 |
| rs13281615 | 8 | A/G | 0.43 | 1.06 | 1.05 | AA | 0.95 | AG | 1.01 | GG | 1.07 |
| rs11780156 | 8 | C/T | 0.052 | 0.84 | 0.98 | CC | 1.02 | CT | 0.85 | TT | 0.72 |
| rs1011970 | 9 | G/T | 0.32 | 1.06 | 1.04 | GG | 0.96 | GT | 1.02 | TT | 1.08 |
| rs10759243 | 9 | C/A | 0.59 | 1.02 | 1.02 | CC | 0.98 | CA | 1.00 | AA | 1.02 |
| rs865686 | 9 | T/G | 0.51 | 1.09 | 1.09 | TT | 0.91 | TG | 1.00 | GG | 1.09 |
| rs2380205 | 10 | C/T | 0.42 | 0.98 | 0.98 | CC | 1.02 | CT | 1.00 | TT | 0.98 |
| rs7072776 | 10 | G/A | 0.49 | 1.04 | 1.04 | GG | 0.96 | GA | 1.00 | AA | 1.04 |
| rs11814448 | 10 | A/C | 0.61 | 1.04 | 1.05 | AA | 0.95 | AC | 0.99 | CC | 1.03 |
| rs10822013 | 10 | T/C | 0.23 | 1 | 1.00 | TT | 1.00 | TC | 1.00 | CC | 1.00 |
| rs10995190 | 10 | G/A | 0.83 | 0.98 | 0.97 | GG | 1.03 | GA | 1.01 | AA | 0.99 |
| rs704010 | 10 | C/T | 0.11 | 0.98 | 1.00 | CC | 1.00 | CT | 0.98 | TT | 0.96 |
| rs7904519 | 10 | A/G | 0.78 | 1.13 | 1.21 | AA | 0.82 | AG | 0.93 | GG | 1.05 |
| rs2981579 | 10 | G/A | 0.59 | 1.18 | 1.22 | GG | 0.82 | GA | 0.96 | AA | 1.14 |
| rs2981582 | 10 | G/A | 0.49 | 1.05 | 1.05 | GG | 0.95 | GA | 1.00 | AA | 1.05 |
| rs11199914 | 10 | C/T | 0.48 | 0.97 | 0.97 | CC | 1.03 | CT | 1.00 | TT | 0.97 |
| rs3817198 | 11 | T/C | 0.17 | 0.98 | 0.99 | TT | 1.01 | TC | 0.99 | CC | 0.97 |
| rs3903072 | 11 | G/T | 0.82 | 0.99 | 0.98 | GG | 1.02 | GT | 1.01 | TT | 1.00 |
| rs554219 | 11 | C/G | 0.22 | 1 | 1.00 | CC | 1.00 | CG | 1.00 | GG | 1.00 |
| rs614367 | 11 | G/A | 0.13 | 0.96 | 0.99 | GG | 1.01 | GA | 0.97 | AA | 0.93 |
| rs7595166 | 11 | C/A | 0.015 | 1.44 | 1.01 | CC | 0.99 | CA | 1.42 | AA | 2.05 |
| rs11820646 | 11 | C/T | 0.78 | 0.98 | 0.97 | CC | 1.03 | CT | 1.01 | TT | 0.99 |
| rs12422552 | 12 | G/C | 0.41 | 1.02 | 1.02 | GG | 0.98 | GC | 1.00 | CC | 1.02 |
| rs10771399 | 12 | A/G | 0.96 | 1.19 | 1.40 | AA | 0.72 | AG | 0.85 | GG | 1.01 |
| rs17356907 | 12 | A/G | 0.79 | 1.02 | 1.03 | AA | 0.97 | AG | 0.99 | GG | 1.01 |
| rs1292011 | 12 | A/G | 0.55 | 1.03 | 1.03 | AA | 0.97 | AG | 1.00 | GG | 1.03 |
| rs11571833 | 13 | A/T | 0.003 | 0.95 | 1.00 | AA | 1.00 | AT | 0.95 | TT | 0.90 |
| rs2236007 | 14 | G/A | 0.93 | 0.9 | 0.82 | GG | 1.22 | GA | 1.09 | AA | 0.98 |
| rs999737 | 14 | C/T | 0.95 | 1.03 | 1.06 | CC | 0.95 | CT | 0.97 | TT | 1.00 |
| rs2588809 | 14 | C/T | 0.29 | 1.01 | 1.01 | CC | 0.99 | CT | 1.00 | TT | 1.01 |
| rs941764 | 14 | A/G | 0.7 | 1.1 | 1.14 | AA | 0.87 | AG | 0.96 | GG | 1.06 |
| rs3803662 | 16 | G/A | 0.51 | 0.99 | 0.99 | GG | 1.01 | GA | 1.00 | AA | 0.99 |
| rs17817449 | 16 | T/G | 0.6 | 1.05 | 1.06 | TT | 0.94 | TG | 0.99 | GG | 1.04 |
| rs11075995 | 16 | A/T | 0.18 | 1.07 | 1.03 | AA | 0.98 | AT | 1.04 | TT | 1.12 |
| rs13329835 | 16 | A/G | 0.63 | 1.08 | 1.10 | AA | 0.91 | AG | 0.98 | GG | 1.06 |
| rs6504950 | 17 | G/A | 0.65 | 1.06 | 1.08 | GG | 0.93 | GA | 0.98 | AA | 1.04 |
| rs527616 | 18 | G/C | 0.86 | 0.98 | 0.97 | GG | 1.04 | GC | 1.01 | CC | 0.99 |
| rs1436904 | 18 | T/G | 0.75 | 0.98 | 0.97 | TT | 1.03 | TG | 1.01 | GG | 0.99 |
| rs8170 | 19 | G/A | 0.19 | 1.13 | 1.05 | GG | 0.95 | GA | 1.08 | AA | 1.22 |
| rs4808801 | 19 | A/G | 0.33 | 1.01 | 1.01 | AA | 0.99 | AG | 1.00 | GG | 1.01 |
| rs3760982 | 19 | G/A | 0.47 | 1 | 1.00 | GG | 1.00 | GA | 1.00 | AA | 1.00 |
| rs2284378 | 20 | C/T | 0.16 | 1.06 | 1.02 | CC | 0.98 | CT | 1.04 | TT | 1.10 |
| rs2823093 | 21 | G/A | 0.57 | 1.03 | 1.03 | GG | 0.97 | GA | 1.00 | AA | 1.03 |
| rs132390 | 22 | T/C | 0.052 | 0.88 | 0.99 | TT | 1.01 | TC | 0.89 | CC | 0.78 |
| rs6001930 | 22 | T/C | 0.13 | 1.02 | 1.01 | TT | 0.99 | TC | 1.01 | CC | 1.04 |

TABLE 11
Hispanic SNPs (n = 82). Alleles represented as major/
minor (eg for rs616488 A is the common allele and G less
common). OR minor allele numbers below 1 means the minor
allele is not the risk allele, whereas when above 1 the
minor allele is the risk allele.

| | | | Minor | OR | | | | | | | |
|---|---|---|---|---|---|---|---|---|---|---|---|
| | | | allele | Minor | | | | | | | |
| SNP | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.0661 | 1.05 | AA | 0.95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | CC | 1.07 |
| rs7696175 | 4 | T/C | 0.38 | 1.14 | 1.11 | TT | 0.90 | CT | 1.03 | CC | 1.17 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs2067980 | 5 | G/A | 0.16 | 1 | 1.00 | GG | 1.00 | AG | 1.00 | AA | 1.00 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs2180341 | 6 | G/A | 0.23 | 0.9600 | 0.98 | GG | 1.02 | AG | 0.98 | AA | 0.94 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs17157903 | 7 | T/C | 0.09 | 0.93 | 0.99 | TT | 1.01 | CT | 0.94 | CC | 0.88 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs2981582 | 10 | T/C | 0.42 | 1.1900 | 1.17 | TT | 0.86 | CT | 1.02 | CC | 1.21 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rs132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

### EXAMPLES

### Example 1—Risk Thresholds

Breast cancer risk assessment is important as it allows the identification of women who are at elevated risk who may benefit from either targeted screening or preventative measures (De la Cruz, 2014; Advani and Morena-Aspitia, 2014). Both genetic and environmental factors are thought to play a role in multifactorial susceptibility to breast cancer (Lichtenstein et al., 2000; Mahoney et al., 2008). In order to optimally assess risk, both components are considered together. Currently, breast cancer risk is often assessed by utilizing the National Cancer Institute's (NCI) Breast Cancer Risk Assessment Tool (BCRAT), often referred to as the “Gail Model” (Gail et al., 1989; Costantino et al., 1999; Rockhill et al., 2001). The BCRAT incorporates several risk factors related to personal history and also incorporates some family history information.

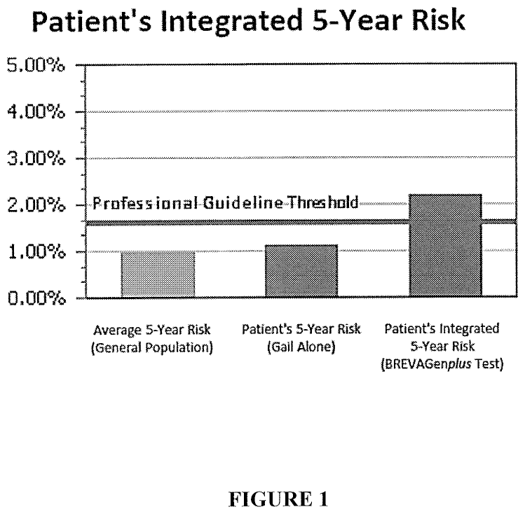

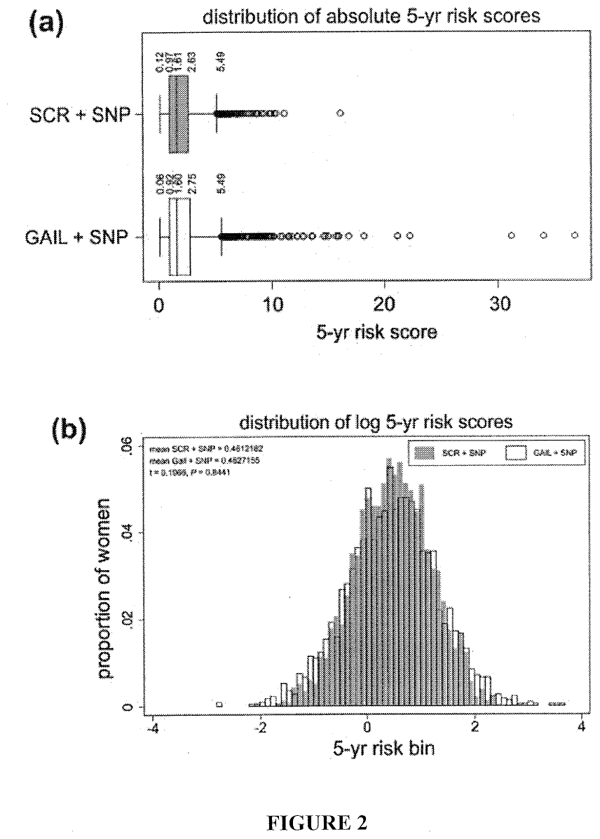

The current model takes the information provided by the ordering physician to calculate a Gail score, and combines it with the patient's common genetic markers for breast cancer to produce Integrated Lifetime and 5-Year patient risk (Example shown in FIG. 1) assessments for breast cancer. It is recommended that a patient receive appropriate genetic or clinical counseling to explain the implications of the test results. American Cancer Society (ACS) guidelines recommend screening breast MRI and mammography for women at high risk (20% lifetime risk). American Society of Clinical Oncology (ASCO) suggest women at high risk (GAIL index>1.66% for 5-year risk) may be offered an estrogen receptor therapy to reduce their risk.

The genetic risk assessment provides additional important information about a woman's risk of developing breast cancer by assessing genetic information from a cheek cell sample. The test detects SNPs. These distinct genetic locations are analysed (genotyped), each of which has been shown reproducibly to modify an individual's odds of developing breast cancer. Scientific validation studies support a simple multiplicative model for combining the SNP risks (Mealiffe et al., 2010).

### Example 2—Combination of SNP Risk Scores with Selected Clinical Information

There are several popular breast cancer risk prediction models. These include BOADICEA (Antoniou et al., 2008 and 2009) and BRCAPRO (Chen et al., 2004; Mazzola et al., 2014; Parmigianin et al., 1998), both of which are based on pedigree data for breast and ovarian cancer; the Gail Model (BCRAT) (Costantino et al., 1999; Gail et al., 1989), which is based on established risk factors for breast cancer and family history represented by the number of first-degree relatives with breast cancer; and the Tyrer-Cuzick Model (IBIS) (Tyrer et al., 2004), which combines information on familial and personal risk factors for breast cancer.

Data points entered into risk prediction algorithms should be as objective as possible to limit ‘noise’ and tighten confidence in the test. While SNPs are an objective measure, patients generally self-report risk factors for the above referenced clinical assessments.

A performance improvement study was designed to (1) identify and confirm the consistency and reliability of self-reported risk factors within clinical laboratory samples and (2) validate a test (‘enhanced’ test) that would use only the most reliable of the self-reported risk factors in combination with SNP profiling.

Missing or “unknown” information entered into Gail model questions were obtained from de-identified test request forms from 2,282 African American, Caucasian, and Hispanic US women who had previously been tested with the BREVAGenplus (Phenogen Sciences) commercial breast cancer risk assessment test.

Table 12 presents the data from the performance improvement study. Approximately 16% (n=2,339) of the Gail-specific information was missing (or answered as unknown). The most commonly missing information was age of menarche, with 4.4% of women who had completed a Gail model questionnaire being unable to provide an answer. The question with the second most common missing (or unknown) information related to whether the patient had at least one biopsy with atypical hyperplasia. There was less than 4% missing information for the other Gail questions, with patient age and ethnicity having no missing information (Table 12).

Missing information may occur because some questions are reliant on memory from decades' past (first menses), while others require a level of medical sophistication on the part of the patient and/or actual pathology reports (atypical hyperplasia). Furthermore, for those who did enter data rather than ‘unknown’, it brings in to question the accuracy of data that is entered into the algorithm. For example, whether or not atypical hyperplasia was present is an important factor in breast cancer risk assessment (Relative Risk >4.0).

TABLE 12
Missing data from Gail Model risk factor fields

| | % (n) of fields |
|---|---|
| | with missing |
| Gail Model Input | information |
| Patient Age | 0.0% |
| Age at menarche | 4.4% |
| Age at time of first live birth | 1.3% |
| First degree relative with Breast Ca | 2.7% |
| Ever had a breast biopsy | 1.1% |
| How many breast biopsies | 2.4% |
| At least one biopsy with atypical hyperplasia | 4.0% |
| Ethnicity | 0.0% |
| Total fields with missing information | 15.9% |

Incomplete data, or most certainly incorrect data, will affect the performance of the risk assessment score if all the Gail fields are used in a clinical risk assessment.

To overcome the limitations associated with patient's entering missing/unknown data for some Gail questions, a revised model was adopted which requires only the patient's age and family history of breast cancer. This model is referred to as the simple clinical risk (SCR) model.

A comparison of risk estimates between the Gail model plus SNP risk versus the SCR model plus SNP risk was performed on 2,282 women that had undergone BREVAGenplus risk assessment and for whom the family history question was complete.

A 5-year absolute clinical risk of breast cancer was created based on published relative risk values for having an affected first-degree relative and taking into account the competing risk of dying from other causes apart from breast cancer. Ethnic-specific breast cancer incidence and competing mortality data were derived from the US SEER database (SEER 2013 Research Data).

SNP-based (relative) risk score was calculated using estimates of the odds ratio (OR) per allele and risk allele frequency (p) assuming independent and additive risks on the log OR scale. For each SNP, the unsealed population average risk was calculated as μ=(1−p)²+2p(1−p)OR+p²OR². Adjusted risk values (with a population average risk equal to 1 were calculated as 1/μ, OR/μ and OR²/μ for the three genotypes defined by number of risk alleles (0, 1, or 2). The overall SNP-based risk score was then calculated by multiplying the adjusted risk values for each of the 77 SNPs.

The clinical model risk scores, the SNP-based score based on the published estimates, and the combined risk scores were log transformed for all analyses. Logistic regression was used to estimate the odds ratio per adjusted standard deviation for log-transformed age-adjusted 5-year risks, Comparative analyses of 5-year risk estimates between the Gail model plus SNP risk versus the SCR model plus SNP risk was performed on the 2,282 patient samples stated above (excluding those patients for whom the 1st degree relative response was missing or unknown), and using a two-sided student's t-test.

The discriminatory power of the SCR model alone, SNP risk alone, and the SCR model with SNP risk was performed on 1,150 Caucasian women, and 7,539 African American and 3,363 Hispanic women, using the area under the receiver operating characteristic curve (AUC) as previously described (Allman et al., 2015; Dite et al., 2016).

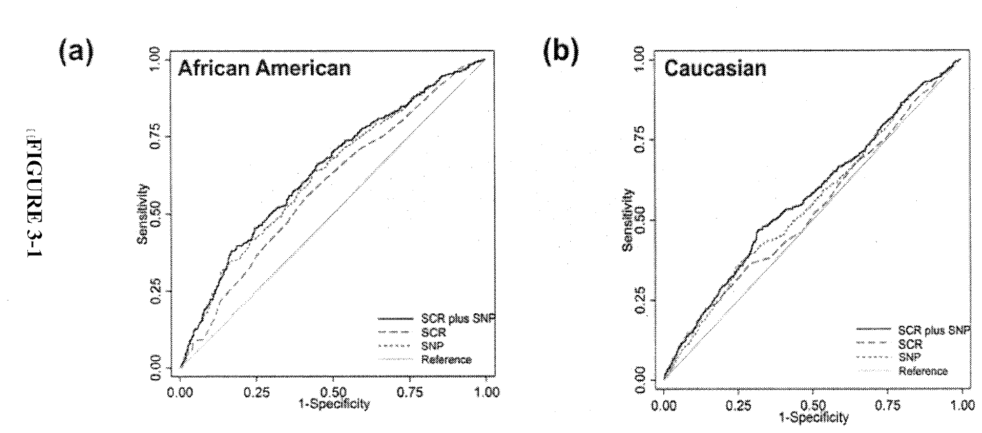

The absolute 5-year risk distribution of the Gail model plus SNP risk (median score of 1.60, FIG. 2a) is very similar to the absolute 5-year risk distribution of the SCR model plus SNP risk (median score of 1.61, FIG. 2a), although the Gail model plus SNP risk had a wider range of risk scores (FIG. 2a). A two-tailed t-test indicates that there is no significant (P=0.8441) difference in mean risk scores between each model (FIG. 2b). This suggests that the reduction in the amount of clinical information used in the SCR model is not having a significantly large effect on breast cancer risk assessment compared to the Gail model.

These data suggest that truncating the clinical information to only two clinical variables does not harm the integrity of the algorithm and this much simpler questionnaire makes it much easier for physicians to record patient data accurately and efficiently. This improved and more efficient patient throughput is important because current United States Preventive Services Task Force (USPSTF) recommendations for risk reduction suggest that ALL women should have a breast cancer risk assessment if they do not risk out based on family history or other high risk factors (such as exposure to radiation).

The American Society of Clinical Oncology defines high-risk women as those having a 5-year risk of 1.67% or more, while the USPSTF uses a 3% threshold to define high-risk women (Visvanathan et al., 2009; Moyer et al., 2013). The present analysis of 2,282 US African American, Caucasian, and Hispanic women revealed that 48.2% of patients exceeded the 1.67% 5-year high-risk threshold, and 21.9% exceeded the 3.0% 5-year high-risk threshold, when using the Gail model and SNP risk (data not shown). Similarly, 48.2% and 18.8% of the 2,282 African American, Caucasian, and Hispanic women were classified as high risk using the SCR model and SNP risk for the 1.67% and 3.% thresholds, respectively. These findings reiterate the importance of more efficient patient throughput due to large numbers of people in which screening is warranted.

### Example 3—Validation of Improved Risk Assessment

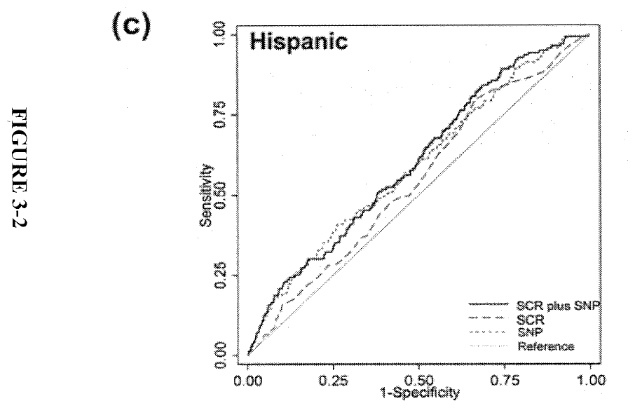

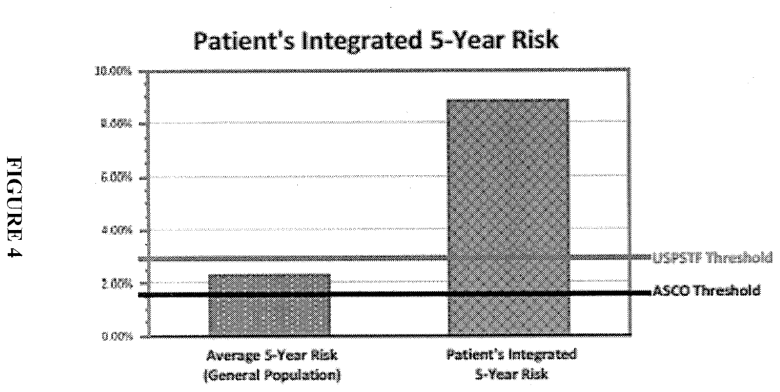

A ROC analysis was carried out to determine whether adding SNP risk to the SCR model predictions improve breast cancer prediction compared to predictions using the SCR model alone. The AUCs were 0.55 (95% CI=0.53, 0.58) for African Americans, 0.61 (95% CI=0.58, 0.65) for Caucasians, and 0.59 (95% CI=0.54, 0.64) for Hispanics when using only SNPs for risk prediction (Table 13). The AUCs were 0.53 (95% CI=0.50, 0.56) for African Americans, 0.59 (95% CI=0.55, 0.62) for Caucasians, and 0.55 (95% CI=0.50, 0.59) for Hispanics when using only the SCR for risk prediction. However, AUCs were highest for risk prediction when using the SCR model in conjunction with SNP risk, with values of 0.57 (95% CI=0.54, 0.60) for African Americans, 0.64 (95% CI=0.61, 0.67) for Caucasians, and 0.60 (95% CI=0.55, 0.65) for Hispanics (Table 13). Thus, ROC analysis confirmed that the SCR model combined with SNP risk gave greater discrimination compared to using the SCR model alone in African American (FIG. 3a), Caucasian (FIG. 3b), and Hispanic (FIG. 3c) women.

TABLE 13
AUC and 95% confidence intervals (CI) in
risk prediction using different models.

| Log-Transformed | | |
|---|---|---|
| Risk Score | AUC | (95% CI) |
| Caucasian (n = 1,155) | | |
| SNP risk only | 0.61 | (0.58, 0.65) |
| SCR model only | 0.59 | (0.55, 0.62) |
| SCR & SNP risk | 0.64 | (0.61, 0.67) |
| African American (n = 7,470) | | |
| SNP risk only | 0.55 | (0.53, 0.58) |
| SCR model only | 0.53 | (0.50, 0.56) |
| SCR & SNP risk | 0.57 | (0.54, 0.60) |
| Hispanic (n = 3,348) | | |
| SNP risk only | 0.59 | (0.54, 0.64) |
| SCR model only | 0.55 | (0.50, 0.59) |
| SCR & SNP risk | 0.60 | (0.55, 0.65) |

The positive likelihood ratio (LR) is the likelihood that a woman with a positive test result would develop breast cancer. As a further measure of the ability of the SCR model plus SNP risk to predict breast cancer risk, positive likelihood ratios were calculated using the 3% USPSTF high-risk threshold as the threshold for a positive breast cancer prediction. African American, Caucasian, and Hispanic are 1.51-, 2.69-, and 2.56-times more likely, respectively to develop breast cancer if they have a positive test result. Positive likelihood ratios were calculated as Sensitivity/1-Specificity using 3.0% 5-year risk as a threshold.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU 2017900208 filed 24 Jan. 2017, the disclosure of which is incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Advani and Morena-Aspitia (2014) Breast Cancer: Targets & Therapy; 6: 59-71
Allman et al. (2015) Breast Cancer Res Treat. 154: 583-9.
Antoniou et al. (2008) Br J Cancer. 98: 1457-1466.
Antoniou et al. (2009) Hum Mol Genet 18: 4442-4456.
American Cancer Society: (2013) Breast Cancer Facts & Figures 2013-1014. Atlanta (Ga.), American Cancer Society Inc, 12.
Cancer, Collaborative Group on Hormonal Factors in Breast Cancer (CGoHFiB) (2001) The Lancet. 358:1389-1399.
Chee et al. (1996) Science 274:610-614.
Chen et al. (2004) Stat Appl Genet Mol Biol. 3: Article 21.
Costantino et al. (1999) J Natl Cancer Inst 91:1541-1548.
De la Cruz (2014) Prim Care Clin Office Pract; 41: 283-306.
Devlin and Risch (1995) Genomics. 29: 311-322.
Dite et al. (2016) Cancer Epidemiol Biomarkers. 154: 583-9.
Fodor (1997a) FASEB Journal 11:A879.
Fodor (1997b) Science 277: 393-395.
Gail et al. (1989) J Natl Cancer Inst 81:1879-1886.
Hartl et al. (1981) A Primer of Population Genetics Washington University, Saint Louis Sinauer Associates, Inc. Sunderland, Mass. ISBN: 0-087893-271-2.
Lichtenstein et al. (2000) NEJM 343: 78-85.
Lockhart (1998) Nature Medicine 4:1235-1236.
Lynch and Walsh (1998) Genetics and Analysis of Quantitative Traits, Sinauer Associates, Inc. Sunderland Mass. ISBN 0-87893-481-2.
Mahoney et al. (2008) Cancer J Clin; 58: 347-371.
Mazzola et al. (2014) Cancer Epidemiol Biomarkers Prev. 23: 1689-1695.
Mealiffe et al. (2010) Natl Cancer Inst. 102: 1618-1627.
Moyer et al. (2013) Ann Intern Med. 159: 698-708.
Parmigiani et al. (1998) Am J Hum Genet. 62: 145-158.
Pencina et al. (2008) Statistics in Medicine 27: 157-172.
Rockhill et al. (2001) J Natl Cancer Inst 93:358-366.
Sapolsky et al. (1999) Genet Anal: Biomolec Engin 14:187-192.
Siegel et al. (2016) Cancer statistics. 66:7-30.
Slatkin and Excoffier (1996) Heredity 76: 377-383.
Sorlie et al. (2001) Proc. Natl. Acad. Sci. 98: 10869-10874.
Tyrer et al. (2004) Stat Med. 23: 1111-1130.
Visvanathan et al. (2009) Journal of Clinical Oncology. 27: 3235-3258.

1. A method for assessing the risk of a human female subject for developing breast cancer comprising:
performing a clinical risk assessment of the female subject, wherein the clinical risk assessment is based only on two or all of the female subject's age, family history of breast cancer and ethnicity;
performing a genetic risk assessment of the female subject, wherein the genetic risk assessment involves detecting, in a biological sample derived from the female subject, the presence of at least two single nucleotide polymorphisms known to be associated with breast cancer; and
combining the clinical risk assessment with the genetic risk assessment to obtain the risk of a human female subject for developing breast cancer.

2. The method of claim 1, which comprises detecting the presence of at least three, five, 10, 20, 30, 40, 50, 60, 70, 80 single nucleotide polymorphisms known to be associated with breast cancer, or wherein the single nucleotide polymorphisms are individually tested for association with breast cancer by logistic regression under a log additive model with no covariates.

3. (canceled)

4. The method according to claim 1, wherein the single nucleotide polymorphisms are selected from: (1) a group consisting of rs2981582, rs3803662, rs889312, rs13387042, rs13281615, rs4415084, rs3817198, rs4973768, rs6504950 and rs11249433, or a single nucleotide polymorphism in linkage disequilibrium with one or more thereof or (2) Table 6 or a single nucleotide polymorphism in linkage disequilibrium with one or more thereof.

5. (canceled)

6. The method according to claim 1, which comprises detecting at least 72 single nucleotide polymorphisms associated with breast cancer, wherein at least 67 of the single nucleotide polymorphisms are selected from Table 7, or a single nucleotide polymorphism in linkage disequilibrium with one or more thereof, and the remaining single nucleotide polymorphisms are selected from Table 6, or a single nucleotide polymorphism in linkage disequilibrium with one or more thereof.

7. The method according to claim 1, wherein when the female subject is Caucasian, the method comprises detecting: (1) at least 72 single nucleotide polymorphisms shown in Table 9, or a single nucleotide polymorphism in linkage disequilibrium with one or more thereof or (2) at least the 77 single nucleotide polymorphisms shown in Table 9, or a single nucleotide polymorphism in linkage disequilibrium with one or more thereof, or
wherein when the female subject is Negroid or African-American, the method comprises detecting: (1) at least 74 single nucleotide polymorphisms shown in Table 10, or a single nucleotide polymorphism in linkage disequilibrium with one or more thereof or (2) at least the 78 single nucleotide polymorphisms shown in Table 10, or a single nucleotide polymorphism in linkage disequilibrium with one or more thereof; or
wherein when the female subject is Hispanic, the method comprises detecting: (1) at least 78 single nucleotide polymorphisms shown in Table 11, or a single nucleotide polymorphism in linkage disequilibrium with one or more thereof or (2) at least the 82 single nucleotide polymorphisms shown in Table 11, or a single nucleotide polymorphism in linkage disequilibrium with one or more thereof.

8-12. (canceled)

13. The method according to claim 1, wherein the results of the clinical risk assessment indicate that the female subject should be subjected to more frequent screening and/or prophylactic anti-breast cancer therapy, or wherein if it is determined the subject has a risk of developing breast cancer, the subject is more likely to be responsive to estrogen inhibition therapy than non-responsive.

14. (canceled)

15. The method according to claim 1, wherein the breast cancer is estrogen receptive positive or estrogen receptor negative.

16. The method according to claim 1, wherein the clinical risk assessment is based only on the female subject's age and family history of breast cancer.

17. The method according to claim 1, wherein combining the clinical risk assessment with the genetic risk assessment comprises multiplying the risk assessments to provide the risk score or involves using the following formula:
abs_risk=mortsuv(1−exp(−RR×SNP(incid_5−incid_age)))
Where RR=the relative risk associated with having a first degree relative with breast cancer, SNP is the composite SNP relative risk, incid_age is the breast cancer incidence at the current (baseline) age, incid_5 is the breast cancer incidence at baseline+5 years, mortsurv is the competing mortality due to causes other than breast cancer.

18. (canceled)

19. A method for determining the need for routine diagnostic testing of a human female subject for breast cancer comprising assessing the risk of the subject for developing breast cancer using the method according to claim 1.

20. The method of claim 19, wherein a risk score greater than about 20% lifetime risk indicates that the subject should be enrolled in a screening breast MRI and mammography program.

21. A method of screening for breast cancer in a human female subject, the method comprising assessing the risk of the subject for developing breast cancer using the method according to claim 1, and routinely screening for breast cancer in the subject if they are assessed as having a risk for developing breast cancer.

22. A method for determining the need of a human female subject for prophylactic anti-breast cancer therapy comprising assessing the risk of the subject for developing breast cancer using the method according to claim 1.

23. The method of claim 22, wherein a risk score greater than about 1.66% 5-year risk indicates that estrogen receptor therapy should be offered to the subject.

24. A method for preventing or reducing the risk of breast cancer in a human female subject, the method comprising assessing the risk of the subject for developing breast cancer using the method according to claim 1, and administering an anti-breast cancer therapy to the subject if they are assessed as having a risk for developing breast cancer.

25. The method of claim 24, wherein the therapy inhibits estrogen.

26. An anti-breast cancer therapy for use in preventing breast cancer in a human female subject at risk thereof, wherein the subject is assessed as having a risk for developing breast cancer according to the method of claim 1.

27. A method for stratifying a group of human female subjects for a clinical trial of a candidate therapy, the method comprising assessing the individual risk of the subjects for developing breast cancer using the method according to claim 1, and using the results of the assessment to select subjects more likely to be responsive to the therapy.

28. A computer implemented method for assessing the risk of a human female subject for developing breast cancer, the method operable in a computing system comprising a processor and a memory, the method comprising:
receiving clinical risk data and genetic risk data for the female subject, wherein the clinical and genetic risk data was obtained by a method according to claim 1;
processing the data to combine the clinical risk data with the genetic risk data to obtain the risk of a human female subject for developing breast cancer;
outputting the risk of a human female subject for developing breast cancer.

29. A system for assessing the risk of a human female subject for developing breast cancer comprising:
system instructions for performing a clinical risk assessment and a genetic risk assessment of the female subject according to claim 1; and
system instructions for combining the clinical risk assessment with the genetic risk assessment to obtain the risk of a human female subject for developing breast cancer.
